(19) United States
(12) Patent Application Publication
(10) Pub. No.: US 2022/0001004 A1
(43) Pub. Date: Jan. 6, 2022

(54) T Cell-Directed Anti-Cancer Vaccines Against Commensal Viruses

(71) Applicant: The General Hospital Corporation, Boston, MA (US)

(72) Inventor: Shadmehr Demehri, Charlestown, MA (US)

(21) Appl. No.: 17/296,829

(22) PCT Filed: Nov. 26, 2019
(86) PCT No.: PCT/US2019/063172

§ 371 Date: May 25, 2021

Related U.S. Application Data
(60) Provisional application No. 62/909,698, filed on Oct. 2, 2019.
(60) Provisional application No. 62/831,691, filed on Apr. 9, 2019.
(60) Provisional application No. 62/772,443, filed on Nov. 28, 2018.

(51) Int. Cl. A61K 39/12; A61P 35/00

(52) U.S. Cl. CPC A61K 39/12; A61K 2039/5258; C12N 2710/20034; A61P 35/00

(57) ABSTRACT

Immune-based approaches to treat and prevent skin cancer by boosting T cell immunity against commensal HPVs present on skin. Thus, provided herein are compositions comprising: (i) a plurality of antigenic peptides each comprising a sequence of 9-30 amino acids derived from proteins from commensal human papilloma viruses, (ii) a plurality of live or live attenuated commensal human papilloma viruses, (iii) a plurality of antigenic proteins from commensal human papilloma viruses, preferably in virus-like particles, and/or (iv) a plurality of nucleic acids encoding (a) a plurality of antigenic peptides, each comprising a sequence of 9-30 amino acids derived from proteins from commensal human papilloma viruses or (b) a plurality of antigenic proteins from commensal human papilloma viruses; and optionally a T cell adjuvant that increases T cell response to the antigenic peptides.

### CLAIM OF PRIORITY

This application claims the benefit of U.S. Patent Application Ser. No. 62/772,443, filed on Nov. 28, 2018; Ser. No. 62/831,691, filed on Apr. 9, 2019; and Ser. No. 62/909,698, filed on Oct. 2, 2019. The entire contents of the foregoing are hereby incorporated by reference.

### FEDERALLY SPONSORED RESEARCH OR DEVELOPMENT

This invention was made with Government support under Grant No. OD021353 awarded by the National Institutes of Health. The Government has certain rights in the invention.

### TECHNICAL FIELD

Immune-based approaches to treat and prevent skin cancer by boosting T cell immunity against commensal HPVs present on skin.

### BACKGROUND

Nonmelanoma skin cancer, including squamous cell carcinoma (SCC) and basal cell carcinoma (BCC), is the most common type of cancer.⁸ Although ultraviolet (UV) radiation is a preventable cause of skin cancer, the incidence of skin cancer in the United States has doubled from 1992 to 2012.⁹ Skin cancers cause significant morbidity including ulceration and disfigurement. Importantly, SCC mortality rate is similar to that of melanoma in immunosuppressed patients including solid organ transplant recipients (OTRs).¹⁰⁻¹² In addition to their side effects, current skin cancer treatments represent a rising public health burden with over $1 billion in total annual cost in the United States.¹³

### SUMMARY

Immunosuppression increases the risk of cancers of viral etiology.¹ Among these, nonmelanoma skin cancer is associated with beta human papillomavirus (β-HPV), particularly in immunosuppressed patients who are at >100-fold increased risk of skin cancer.²⁻⁵ However, previous studies have failed to establish a causative role for low-risk HPVs in skin cancer. Here, we provide an alternative explanation for this association by demonstrating that anti-papillomavirus immunity suppresses skin cancer in immunocompetent hosts: the loss of this immunity rather than the oncogenic effect of commensal HPVs is the reason for markedly increased risk of skin cancer in immunosuppressed patients. In a clinical study, we found that the anatomical distribution of skin cancers in immunosuppressed patients was significantly different from their HPV-driven warts, but matched the distribution of skin cancers in immunocompetent patients. This pattern of skin cancer distribution suggested that the ultraviolet (UV) radiation was the primary cause of cancer in both populations. To experimentally investigate the impact of papillomavirus on carcinogen-driven skin cancer, we colonized immunocompetent wild-type (Wt) C57BL/6, FVB and SKH-1 mice with mouse papillomavirus type 1 (MmuPV1).^{6,7} Colonized mice with natural immunity against MmuPV1 or acquired immunity through T cell transfer from immune mice gained marked protection against chemical- and UV-induced skin carcinogenesis compared to their uninfected counterparts. RNA and DNA in situ hybridizations for 25 commensal β-HPVs revealed a significant loss of viral activity and load in human skin cancer cells compared to the adjacent normal skin. Finally, β-HPV E7 peptides activated CD8⁺ T cells isolated from normal human skin. Our findings reveal a beneficial effect of commensal viruses and establishes the foundation for immune-based approaches to treat and prevent skin cancer by boosting T cell immunity against commensal HPVs present on all of our skin.

Thus, provided herein are compositions comprising: (i) a plurality of antigenic peptides each comprising a sequence of 9-30 amino acids derived from proteins from commensal human papilloma viruses, (ii) a plurality of live or live attenuated commensal human papilloma viruses, (iii) a plurality of antigenic proteins from commensal human papilloma viruses, preferably in virus-like particles, and/or (iv) a plurality of nucleic acids encoding (a) a plurality of antigenic peptides, each comprising a sequence of 9-30 amino acids derived from proteins from commensal human papilloma viruses or (b) a plurality of antigenic proteins from commensal human papilloma viruses; and optionally a T cell adjuvant that increases T cell response to the antigenic peptides. In some embodiments, the commensal human papilloma viruses are low risk α-HPV, β-HPV, γ-HPV, and/or μ-HPV strains, e.g., the commensal human papilloma viruses are low risk α-HPV, β-HPV, γ-HPV, and/or μ-HPV strains listed in Table A.

In some embodiments, the plurality of antigenic peptides comprises peptides derived from one or more E1, E2, E4, E5, E6 or E7 proteins.

In some embodiments, the plurality of antigenic peptides comprises peptides derived from proteins from a plurality of commensal human papilloma viruses.

In some embodiments, the compositions comprise at least 200 peptides each having a unique sequences, e.g., comprising a plurality of peptides for each unique sequence.

In some embodiments, the composition comprises one or more viral vectors engineered to express the plurality of proteins or antigenic peptides, e.g., viral vectors selected from the group consisting of recombinant retroviruses, adenovirus, adeno-associated virus, alphavirus, and lentivirus.

In some embodiments, the T cell adjuvant comprises one or more of nanoparticles that enhance T cell response; poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminium salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant). In some embodiments, the T cell adjuvant comprises topical resiquimod and/or imiquimod and/or topical 5-fluorouracil and/or topical calcipotriene (calcipotriol), e.g., in combination with 5-fluorouracil.

Also provided herein are methods of treating, or reducing the risk of developing, skin cancer in a subject, the method comprising administering to the subject an effective amount of a composition as described herein. Additionally provided are the compositions described herein for use in a method of treating, or reducing the risk of developing, skin cancer in a subject.

In some embodiments, the subject has an increased risk of developing skin cancer or is immunocompromised, e.g., as a result of aging or an acquired immunodeficiency, primary immunodeficiency, or an organ transplant.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-J: MmuPV1 skin colonization protects animals against chemical skin carcinogenesis. A-C, Skin tumor outcomes for DMBA-TPA-treated MmuPV1-colonized wild-type (Wt) C57BL/6J mice (MmuPV1/DMBA-TPA, n=12), DMBA-TPA-treated sham-infected (−/DMBA-TPA, n=10) and MmuPV1-colonized mice (MmuPV1/−, n=10) are assessed by (A) tumor latency, (B) tumor counts per mouse over time, and (C) tumor burden at the completion of the carcinogenesis protocol. D, The percentage of Wt FVB mice with wart development on their back skin immediately after MmuPV1 infection and a subgroup with persistent warts are shown. E, T cells from skin-draining lymph nodes of MmuPV1-colonized immune mice (test T cells) are transferred to those with persistent warts. The changes in skin wart burden is documented at 2 weeks post adoptive T cell transfer. Control T cells represent naïve T cells as found in the spleen of uninfected Wt FVB mice. F-I, Skin tumor development in DMBA-TPA-treated MmuPV1-colonized Wt FVB mice (MmuPV1/DMBA-TPA, n=10) is compared to DMBA-TPA-treated sham-infected (−/DMBA-TPA, n=10) and MmuPV1-colonized mice (MmuPV1/−, n=10). (F) time to tumor onset, (G) number of skin tumors over time, (H) tumor burden at the completion of the study, and (I) representative images of mice in DMBA-TPA-treated cohorts are shown. (J) Mice that rejected their skin warts after receiving T cells from MmuPV1-colonized immune mice (test T cells in e; n=3) compared with wart-bearing non-immune mice (n=3) after treatment with DMBA-TPA. Note that non-immune mice developed invasive skin cancers at 8 weeks (mouse 4), 15 weeks (mouse 5) and 20 weeks (mouse 6) after DMBA-TPA treatment (red circles). Mice were shaved to enable better visualization of the skin tumors. Scale bars: lcm, error bars represent the mean+SD; *p<0.05, **p<0.01, ***p<0.0001.

FIGS. 2A-G: MmuPV1 skin colonization protects immunocompetent SKH-1 mice against UV carcinogenesis. A-C, SKH-1 mice that are colonized with MmuPV1 on their back skin and have no warts (i.e., immune) are subjected to DMBA-UV carcinogenesis protocol. (A) Time to tumor onset, (B) tumor counts per mouse over time, and (C) the number of tumors developed in each mouse over the course of the carcinogenesis protocol are compared between DMBA-UV-treated MmuPV1-colonized SKH-1 mice (MmuPV1/DMBA-UV, n=10), DMBA-TPA-treated sham-infected mice (−/DMBA-UV, n=10) and MmuPV1-colonized mice (MmuPV1/−, n=10). D, Representative images of mice in DMBA-UV-treatment groups are shown (scale bar: lcm). Note the resemblance of DMBA-UV-induced skin tumors specially in −/DMBA-UV cohort to actinic keratosis and SCC in humans. E, Representative images of CD8⁺ T cells in the skin of MmuPV1/DMBA-UV and −/DMBA-UV mice at the completion of carcinogenesis protocol are shown (dashed lines highlight the epidermal basement membrane, scale bar: 100 m). F, CD8⁺ T cell infiltrates in MmuPV1/DMBA-UV and −/DMBA-UV skin are quantified in 10 random high-power field (hpf) per skin and averaged across the mice in each group. G, The ratio of CD8⁺ T cells within the epithelial compartments (i.e., CD8⁺ T_{RM} cells) over the total T cell count in each high-power image is calculated across MmuPV1/DMBA-UV and −/DMBA-UV skin samples and presented as a graph. Stained cells are counted blindly. Each dot represents one high power image. error bars represent the mean+SD; *p<0.05, **p<0.005, ***p<0.001, ns: not significant.

FIGS. 3A-D: A significant fall in β-HPV activity from normal skin to skin cancer and the presence of β-HPV-specific cytotoxic T cells in the normal human skin points to a potent selective pressure by antiviral immunity against malignant cells with active HPV. A, β-HPV RNA in situ hybridization (RNAish) using a pool of probes specific for 25 β-HPV types is used to detect β-HPV transcripts (red dots) in human skin cancers (Table 2). Wart serves as a positive control and exhibits the greatest amount of β-HPV activity. Hypertrophic actinic keratosis arising in association with a wart (HAK in verruca) is another example of a β-HPV-active lesion found on the skin of immunosuppressed patients. Representative RNAish-stained sections of SCC from immunosuppressed and immunocompetent patients are shown. Insets highlight the representative areas of the cancer/wart and their adjacent normal skin (scale bars: 100 m). B, RNAish signals were quantified in paired samples of skin cancer and the adjacent normal skin across skin cancer specimens collected from immunosuppressed (n=38) and immunocompetent patients (n=32, skin cancer characteristics are listed in Table 3). C AND D, Representative flow plots (C) and quantification of activated CD69⁺ and CD137⁺ CD69⁺ cytotoxic T lymphocytes (D) isolated from human facial skin and used in a peptide stimulation assay are shown. Percentage of CD8⁺ T cells in each quadrant is listed on the flow plots. T cells from 8 facial skin samples (6 males and 2 females) are used in this assay (average age: 75, age range: 60-89). Note that β-HPV peptide pool used in this assay is a collection of E7 peptides from 5 β-HPV types (HPV5, 8, 9, 20 and 38) and HPV16 represents a pool of HPV16 E7 peptides (Table 4). PMA/Ionomycin stimulation is used as a positive control. Error bars represent the mean+SD; *p<0.05, **p<0.01, ns: not significant.

FIGS. 4A-B. The divergent anatomical distribution of warts versus skin cancers in immunosuppressed and immunocompetent patients. A, Warts from immunosuppressed patients and skin cancers from immunosuppressed and immunocompetent patients are mapped. Note that Immunocompetent and immunosuppressed patients' skin cancer localizations closely match each other and are almost entirely restricted to chronically sun damaged (CSD)>intermittently sun damaged (ISD) areas of the skin. Warts show anatomic preference away from sun exposure. B, graphs showing anatomical distribution.

FIGS. 5A-B. T cell-deficient mice infected with MmuPV1 on the dorsal skin demonstrate a confluent pattern of wart development. A, Significant wart burden in CD4^{-/-}; CD8^{-/-} mice (right) is compared with no warts in Wt mice (left) following MmuPV1 infection of the dorsal/back skin (10 weeks after infection, scale bar: lcm). B, MmuPV1-induced wart in CD4^{-/-}; CD8^{-/-} mouse stained with H&E (left), MmuPV1 L2 RNAish (middle) and negative control RNAish probe (right; scale bar: 1 mm).

FIGS. 6A-C. MmuPV1 DNA is detected in all skin samples biopsied across Wt animals' dorsal skin after MmuPV1 infection, indicative of virus colonization. A, A representative image of C57BL/6J mice back skin on the day of MmuPV1 infection and 21 days post-infection are shown. Positive PCR bands in all corresponding sections of skin is shown. A typical C57BL/6J mouse 5 weeks post-infection with no evidence of skin wart is also shown. B, Representative images demonstrate the back skin of FVB mice on the day of infection and 31 days post-infection. Positive bands in all corresponding sections of skin is shown. MmuPV1 PCR bands are marked by arrows (PCR amplicon size: 339 bp). C, Left, representative images of the back skin of wild-type C57BL/6J mice on the day of MmuPV1 infection and 21 days after infection. Middle, MmuPV1 L1 PCR on 20 segments of the back skin. MmuPV1 L1 PCR bands are marked by arrows; PCR amplicon size, 339 bp. PCR primers, forward: GAGCTCTTTGTTACTGTTGTC (SEQ ID NO:1); reverse: ATCCTCTCTTTCCTTGGGC (SEQ ID NO:2). M, molecular-weight size marker; N, negative control; P1-P3, positive controls. Right, a typical wild-type C57BL/6J mouse five weeks after infection, highlighting the absence of warts, which was the case for 100% of the mice. Scale bars: lcm.

FIGS. 7A-C. Memory T cells transferred from Wt MmuPV1-colonized mice to T cell-deficient mice reduce the wart burden upon MmuPV1 infection, but have no impact on SCC cell line growth in T cell recipient animals. A, Schematic of T cell transfer experiment. B, Representative images of the warts on the back skin of mice 3 weeks after MmuPV1 infection are shown. Flow cytometry demonstrates the presence of CD4⁺ and CD8⁺ T cells in the peripheral blood of the recipient mice, indicating a successful adoptive T cell transfer. C, Growth of subcutaneously injected SCC cells is monitored in Wt mice, CD4^{-/-}; CD8^{-/-} mice, and CD4^{-/-}; CD8^{-/-} mice that received T cells from MmuPV1-immune donors.

FIGS. 8A-J. Evidence of virus colonization and T cell homing into the epithelium of MmuPV1-infected mice are found at the completion of the chemical carcinogenesis protocol. A and B, MmuPV1 L1 PCR is used to detect viral DNA isolated from the skin of (A) C57BL/6J (B6) and (B) FVB mice >6 months after the infection. C and D, Anti-MmuPV1 seroconversion is assessed in DMBA-TPA-treated cohorts of (C) C57BL/6J and (D) FVB mice. (E), Representative images of CD3/CD45-stained skin from MmuPV1/DMBA-TPA FVB mice compared with sham/DMBA-TPA controls at the completion of the chemical carcinogenesis protocol. Arrows indicate T cells in the epidermis; dashed lines highlight the epidermal basement membrane. (F), CD45+ leukocytes quantified in skin sections of MmuPV1/DMBA-TPA and sham/DMBA-TPA FVB mice across ten randomly selected HPF images of normal skin per mouse and averaged across the mice in each group (two-tailed unpaired t-test; n=8 per group). Each dot represents the leukocyte count in one highpower image. g, h, Homing of T cells to the epidermis in MmuPV1/DMBA-TPA skin compared with sham/DMBA-TPA control skin of wild-type FVB mice. (G), Representative images of CD8/CD3- and CD4/CD3-stained skin sections. Arrows indicate epidermal CD8+ TRM cells; dashed lines highlight the epidermal basement membrane. (H), The ratio of epidermal CD8+ TRM and CD4+ TRM cells to total CD3+ T cells in the skin per HPF image (two-tailed unpaired t-test). T cells in up to ten randomly selected HPF images of normal skin per mouse were counted. Each dot represents one high-power image. n=10 (MmuPV1/DMBA-TPA); n=9 (sham/DMBA-TPA). (I), Representative skin tumors from MmuPV1/DMBA-TPA and sham/DMBA-TPA wild-type FVB mice stained with keratin 6 (K6; a marker for epidermal hyperplasia) and Ki67 (a proliferation marker). Dashed lines highlight the epidermal basement membrane in the skin. (J), PCR amplification of the wildtype (A) and mutant (T) region of the Hras gene in DNA of MmuPV1/DMBA-TPA and sham/DMBA-TPA tumors and skin, and untreated skin from a wild-type FVB mouse (band size, 110 bp). The A-to-T mutation in Hras codon 61 highlights DMBA-TPA-induced skin tumors in MmuPV1/DMBA-TPA and sham/DMBA-TPA wild-type FVB cohorts. Scale bar: 100 m, error bars represent the mean+SD; *p<0.05, **p<0.01, ***p<0.0001, ns: not significant.

FIG. 9. Carcinogen-induced skin tumors in mice lack the viral activity seen in warts. H&E and MmuPV1 RNAish images of a wart from MumPV1-infected CD4^{-/-}; CD8^{-/-} mouse, a skin tumor and normal skin from MmuPV1-colonized DMBA-TPA-treated Wt mouse are shown. Note the dense RNAish signals in the wart from T cell deficient mouse. After the competition of DMBA-TPA treatment, positive MmuPV1 RNAish signals are detected in the normal skin of a Wt mouse. There is minimal MmuPV1 RNAish signal in the skin tumor from the same mouse.

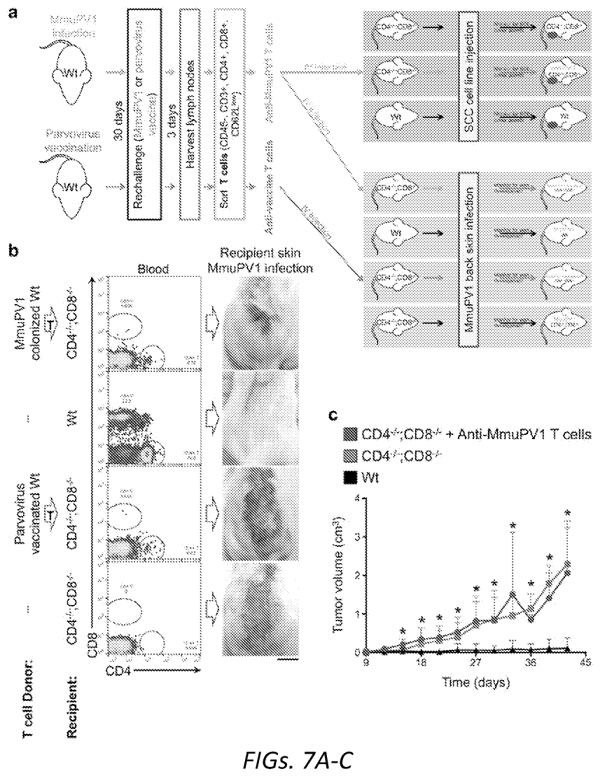

FIGS. 7A-B. MmuPV1 RNAish of MmuPV1-infected mice reveals active virus in both immune and nonimmune mice. A, Representative images of SKH-1 mice with no evidence of disease following infection (immune) and mice with visible warts after dorsal skin infection (nonimmune) are shown (scale bar: 1 cm). B, Viral L2 protein RNAish of an immune mouse and a nonimmune mouse skin that were harvested 3 weeks after MmuPV1 infection shows abundant viral activity in the normal skin and the MmuPV1-driven wart. Insets highlight the areas of active virus in the normal skin of the immune mouse and the wart of the nonimmune mouse (scale bars: 100 m).

FIGS. 10A-M. Immunization of MmuPV1-infected SKH-1 mice with MmuPV1 vaccine protects against UV-driven carcinogenesis. A, Top, representative images of SKH-1 mice with no evidence of disease following infection (immune) and with visible warts after back-skin infection with MmuPV1 (non-immune). Bottom, MmuPV1 L2 RNA ISH of skin from an immune and a nonimmune mouse, collected three weeks after infection with MmuPV1, to detect viral activity in the normal skin and the MmuPV1-driven wart. Insets highlight the active virus in the normal skin of the immune mouse and the wart of the nonimmune mouse. B, Macroscopic images of the SKH-1 mice three months after MmuPV1 back-skin infection. SKH-1 mice with spontaneous immunity to the virus (no wart) were treated once with an immunosuppressive dose of UVB (300 mJ cm−2); images of the mice three weeks after UV treatment are shown. Arrows point to the newly developed warts on the UV-treated skin. C, Histological images of a wart (circle), stained with H&E and MmuPV1 RNA ISH. The magnified inset highlights MmuPV1-induced cytopathic changes in the H&E image and confluent positive MmuPV1 RNA ISH signals in the wart. D, Macroscopic images of MmuPV1-infected SKH-1 mice that continued to have warts (arrows) before MmuPV1 vaccination, four weeks after vaccination and at the completion of the UV carcinogenesis protocol. The nine wart-bearing mice were treated with MmuPV1 live virus particles intraperitoneally three times over two weeks. Four weeks later, the mice underwent the UV carcinogenesis protocol. Mice with acquired antiviral immunity (n=5) are compared with nonimmune mice that have persistent warts (n=4). E, Skin tumor burden in vaccinated immune (n=5) and non-immune (n=4) mice treated with the UV carcinogenesis protocol. In mice with a confluent pattern of skin tumors, counts represent the individual lesions before their coalescence. Two-tailed Mann-Whitney U test; data are mean±s.d. F, Representative images of CD3/CD45-stained skin from MmuPV1/DMBA-UV SKH-1 mice compared with sham/DMBA-UV controls at the completion of the UV carcinogenesis protocol. Arrows indicate T cells in the epidermis; dashed lines highlight the epidermal basement membrane. G-I, Skin-infiltrating total CD45+ leukocytes (G), CD3+CD45+ T cells (H) and CD3-CD45+ leukocytes (I) quantified in CD3/CD45-stained skin sections of MmuPV1/DMBA-UV (n=10) and sham/DMBA-UV (n=9) SKH-1 mice across ten randomly selected HPF images of each skin sample and averaged across the mice in each group. Each dot represents one high-power image. Note the trend towards an increase in T cells and a decrease in CD3− inflammatory cells in MmuPV1/DMBA-UV skin compared with sham/DMBA-UV control. J, Representative images of CD3/CD45-stained cells in the skin tumors of MmuPV1/DMBA-UV SKH-1 mice compared with sham/DMBA-UV controls at the completion of the UV carcinogenesis protocol. Magnified insets highlight the immune cells in the tumor parenchyma. K-M, Tumor-infiltrating total CD45+ leukocytes (K), CD3+CD45+ T cells (L) and CD3-CD45+ leukocytes (M) quantified in CD3/CD45-stained sections of MmuPV1/DMBA-UV and sham/DMBA-UV SKH-1 skin tumors across HPF images of each tumor and averaged across the mice in each group (n=12 early skin tumors per group). Each dot represents one high-power image. Stained cells were counted blindly. Two-tailed unpaired t-test; data are mean+s.d. (G-I, K-M). Scale bars, mouse, 1 cm (A, B, D); tissue, 100 m (A, C, F, J).

FIGS. 11A-N. CD8+ T cell immunity is required to protect MmuPV1-colonized mice from UV carcinogenesis and MmuPV1 colonization protects Xpc−/−mice from UV carcinogenesis. A, Representative images of CD8+ T cells in the skin tumors of MmuPV1/DMBA-UV SKH-1 mice compared with sham/DMBA-UV controls at the completion of the UV carcinogenesis protocol. Magnified insets highlight T cells in the tumor parenchyma. B-D, Tumorinfiltrating CD3+(B), CD8+(C) and CD4+(D) T cells quantified in CD8/CD3- and CD4/CD3-stained tumor sections of MmuPV1/DMBA-UV and sham/DMBA-UV SKH-1 mice across HPF images of each tumor and averaged across the mice in each group (n=12 early skin tumors per group). Each dot represents one highpower image. e, f, CD4+ T cell infiltrates in MmuPV1/DMBA-UV and sham/DMBA-UV SKH-1 skin. E, Representative images of the CD4/CD3-stained skin sections. Arrows indicate epidermal CD4+ TRM cells; dashed lines highlight the epidermal basement membrane, F, Quantification of CD4+ T cells per high-power image of the skin. Ten randomly selected HPF images of skin per mouse in each group are included. Each dot represents one high-power image. n=10 (MmuPV1/DMBA-UV); n=9 (sham/DMBA-UV). Two-tailed unpaired t-test; data are mean+s.d. (B-D, F). G, Schematic diagram of anti-CD8 or IgG antibody treatment combined with the UV carcinogenesis protocol. Four weeks after MmuPV1 or sham(VLP) infection, mice began treatment with anti-CD8 or IgG isotype control antibodies (arrows). A day after the first treatment with antibodies, the back skin of SKH-1 mice was treated with 50 g DMBA once (darker grey triangle). Seven days later, mice began UVB treatment (100 mJ cm-2) three times a week (light greytriangles). H, Flow cytometry analysis of spleen and skin of MmuPV1/DMBA-UV mice treated with anti-CD8 or IgG antibodies to evaluate the efficiency of CD8+ T cell depletion at six weeks after treatment with DMBA. The percentage of CD8+ T cells is shown on each plot. I, Skin tumor burden in MmuPV1-colonized mice treated with IgG control (MmuPV1+IgG; n=10) or anti-CD8 antibody (MmuPV1+anti-CD8; n=10), and sham(VLP)-infected mice treated with IgG control (sham(VLP)+IgG; n=7) or anti-CD8 antibody (sham(VLP)+anti-CD8; n=7) after DMBA-UV treatment. Two-tailed Mann-Whitney U test; *P<0.05, NS, not significant. Data are mean±s.d. J, Representative images of mice in the four treatment groups. Owing to the large skin tumors in MmuPV1-colonized CD8+ T cell-depleted mice, the UV carcinogenesis study was terminated at 18 weeks after DMBA treatment. K, L, Xpc−/−(XPCKO) mice were infected with MmuPV1 on their back skin (n=15) or sham-infected (n=13) and subjected to the UV carcinogenesis protocol. Skin tumor outcomes are shown as the time to development of the first skin tumor (K) and time to development of the first invasive skin cancer (L) (log-rank test). Note that all Xpc−/−mice in the study were immune to MmuPV1 (that is, exhibited no wart development). M, Representative images of Xpc−/−mice at the completion of the 30-week UV carcinogenesis protocol. Premalignant tumors (papillomas) and invasive skin cancers are highlighted with yellow and red circles, respectively. Mice were shaved for UV treatments and the visualization of the skin tumors. N, Representative H&E stained histological images of a papilloma in MmuPV1/DMBA-UV and invasive skin cancer in sham/DMBA-UV Xpc−/−mice. The inset shows the cellular atypia in the sham/DMBA-UV skin cancer (scale bar, 50 m). Stained cells were counted blindly. Scale bars, mouse, 1 cm (j, m); tissue: 100 m (a, e, n).

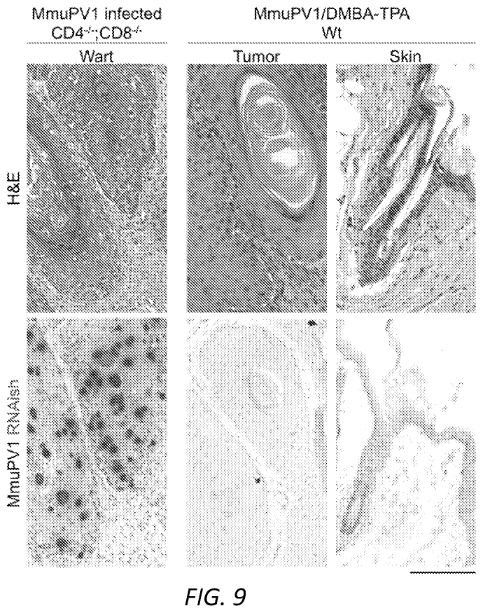

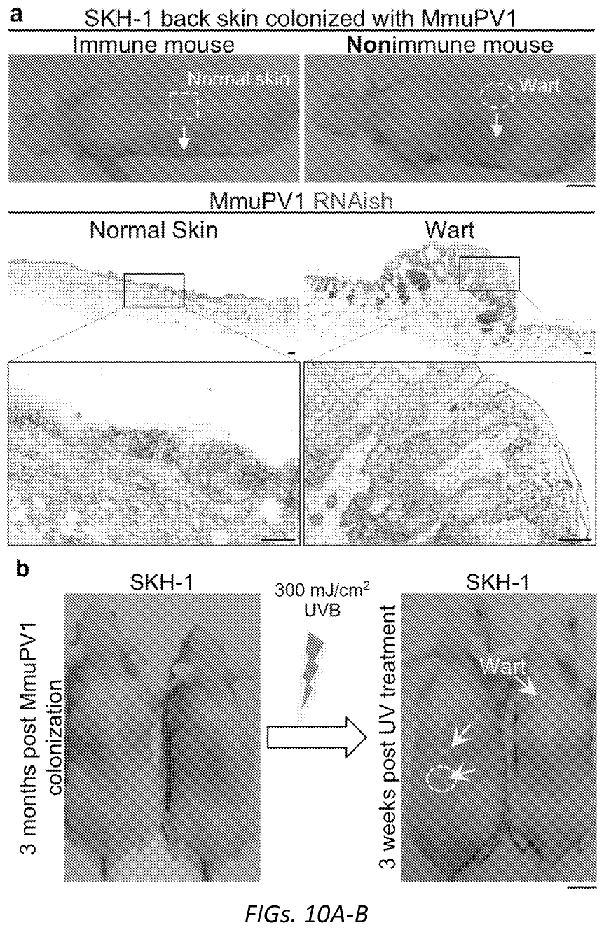

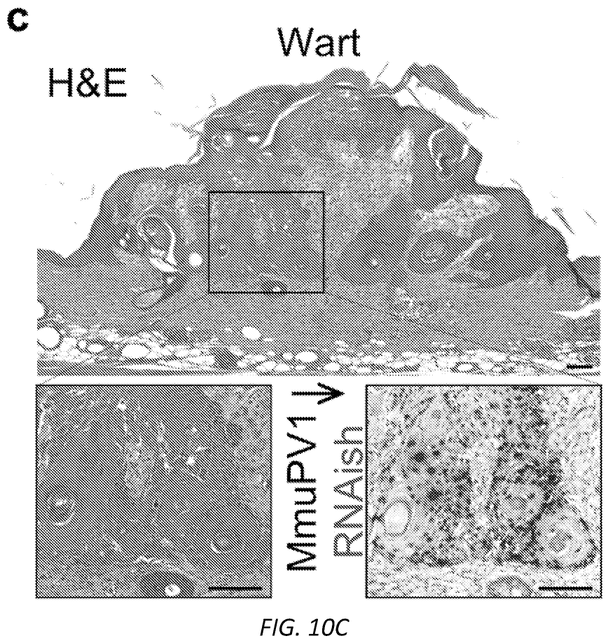

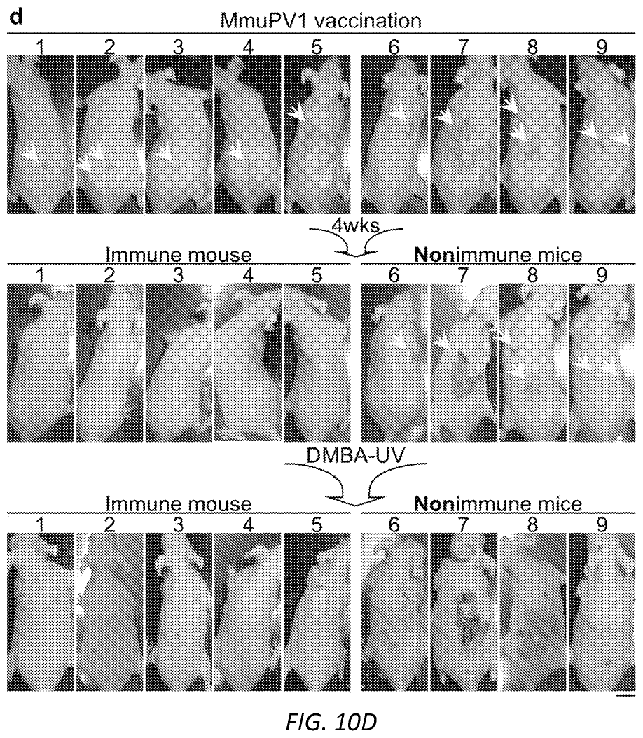

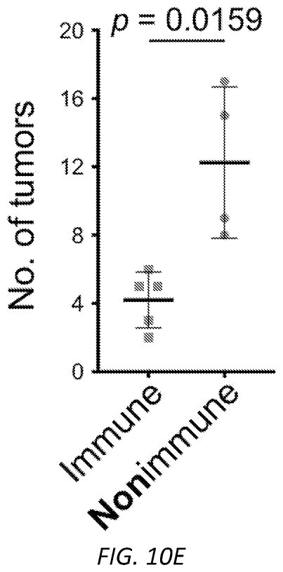

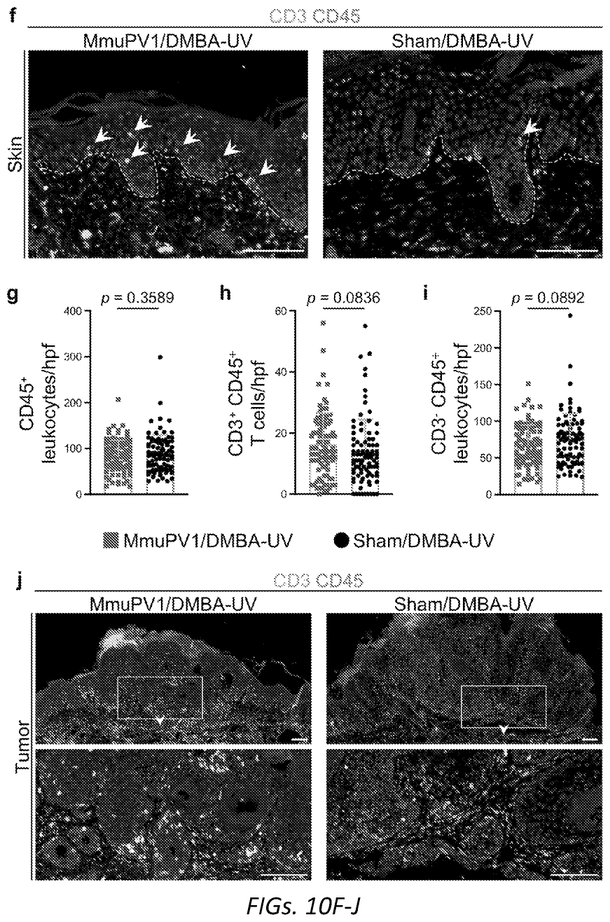

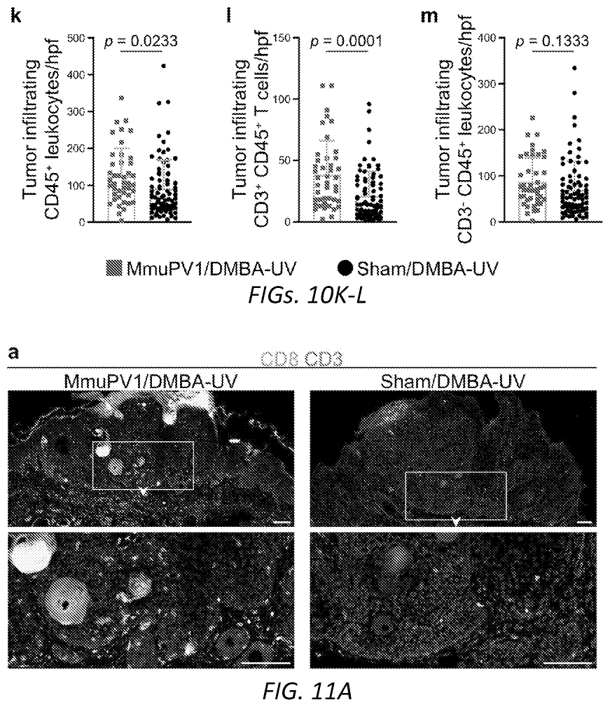

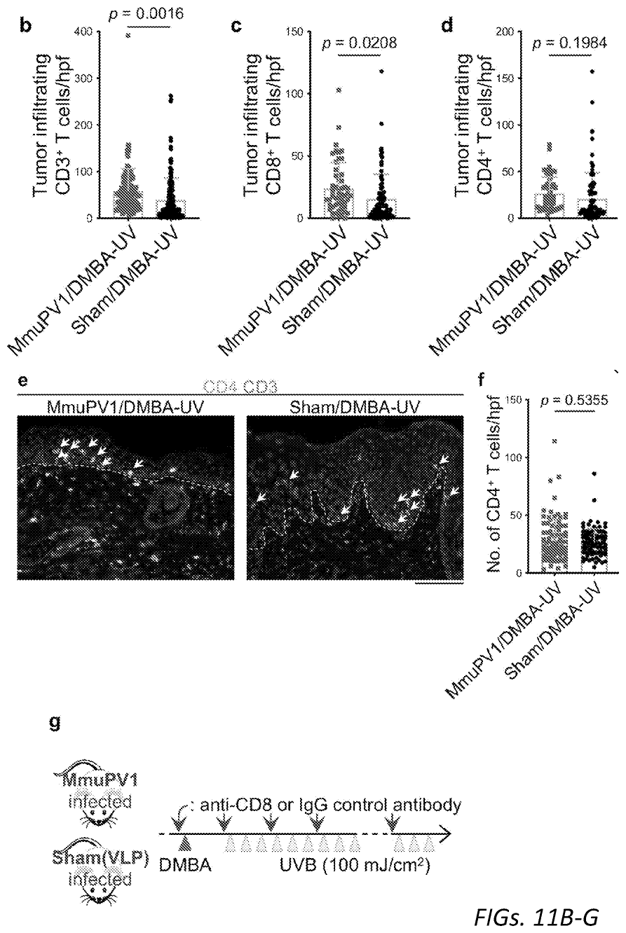

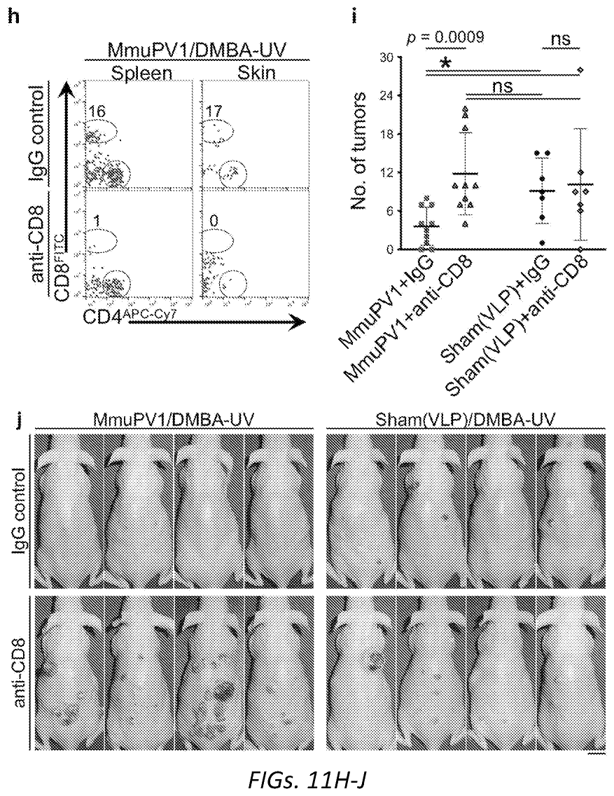

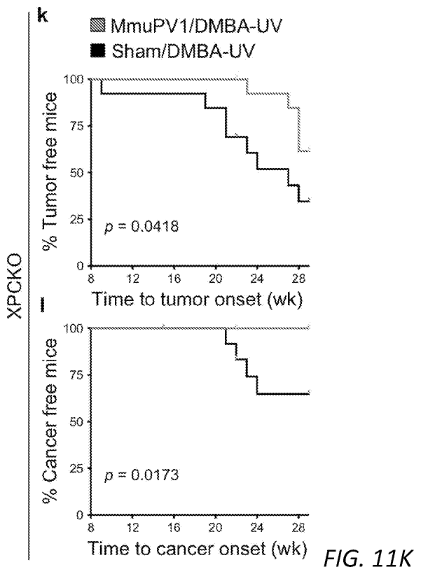

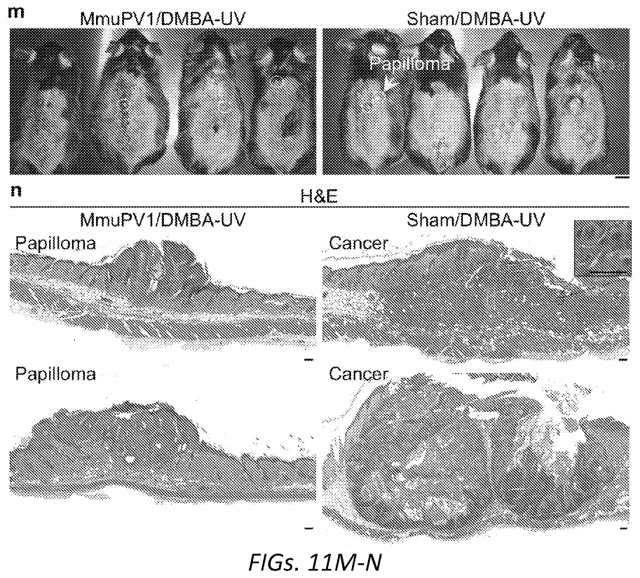

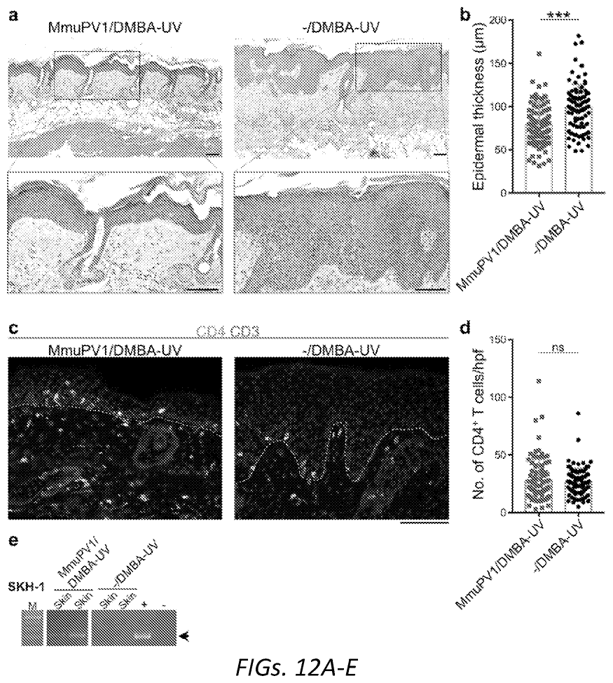

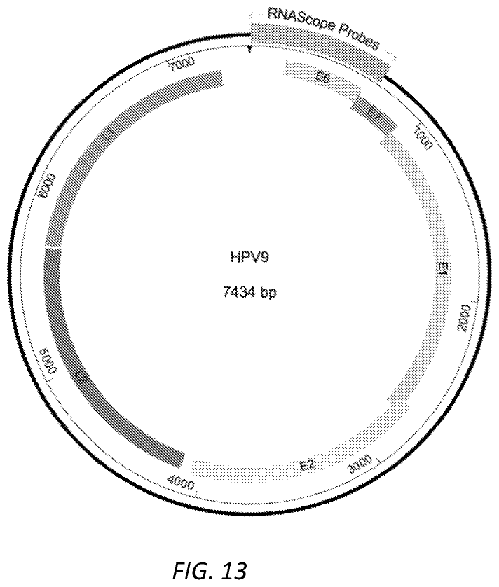

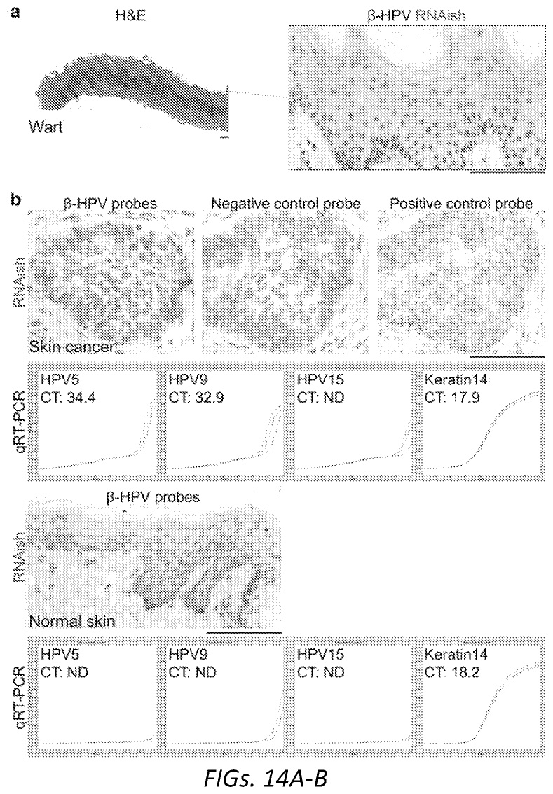

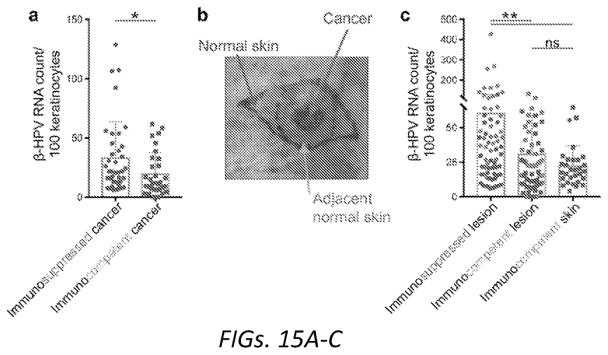

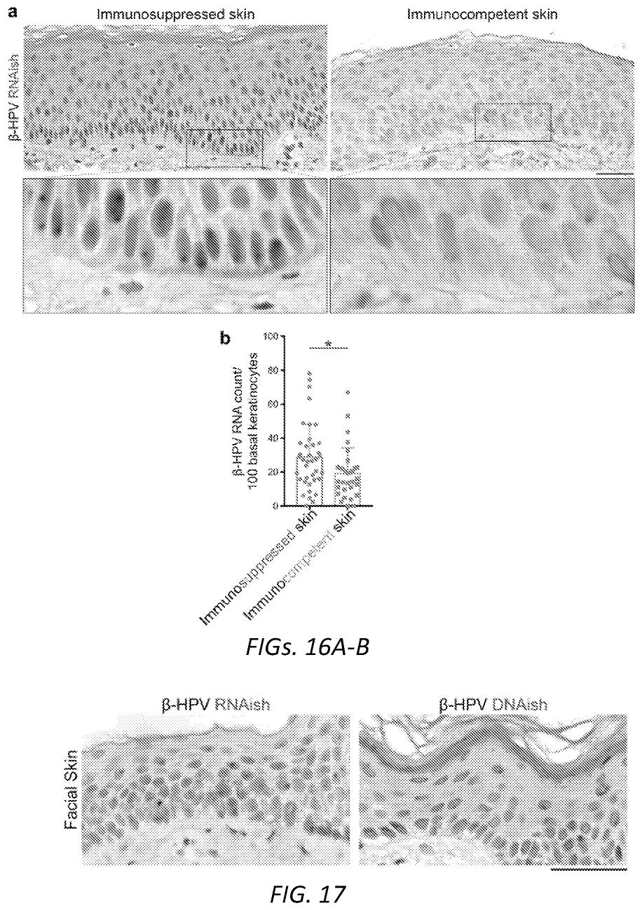

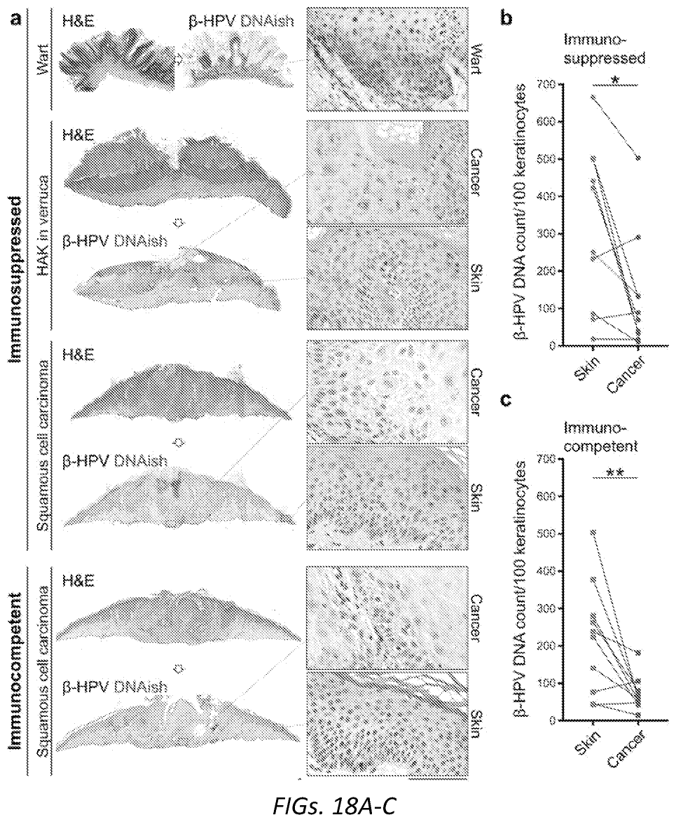

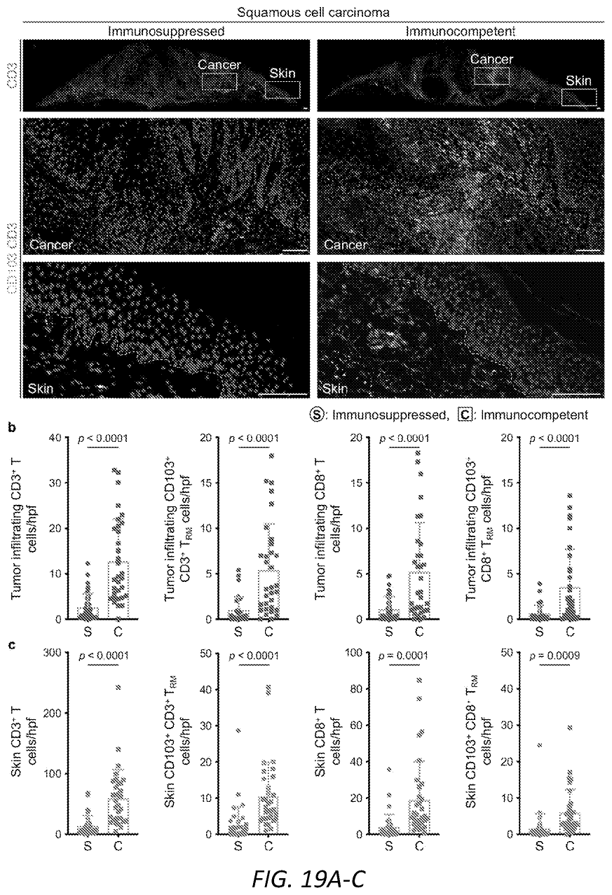

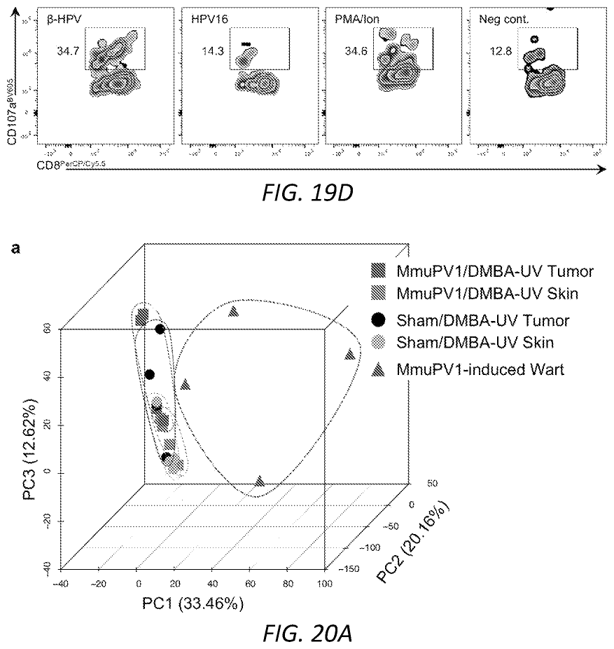

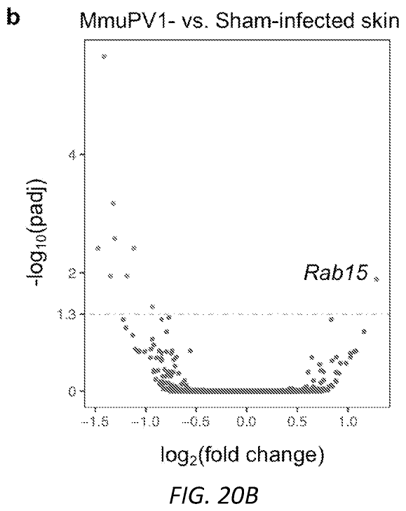

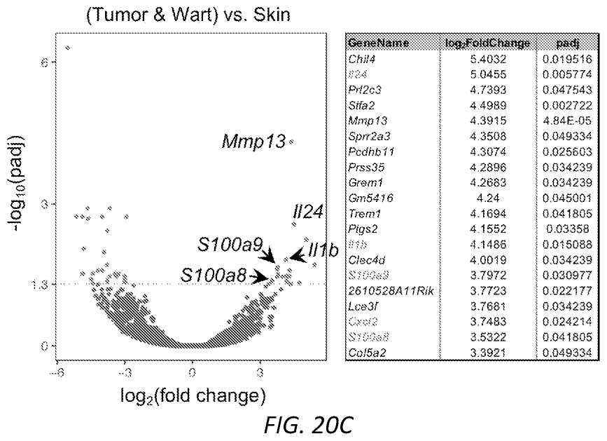

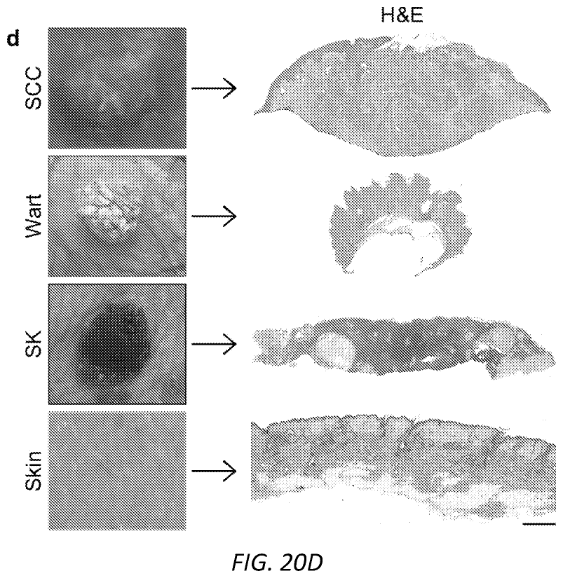

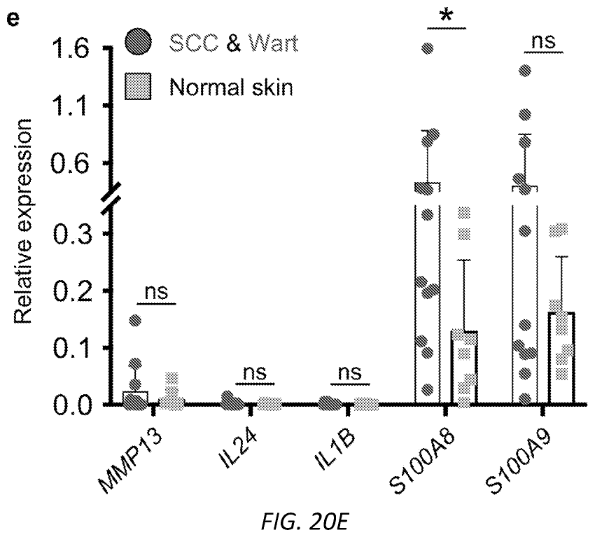

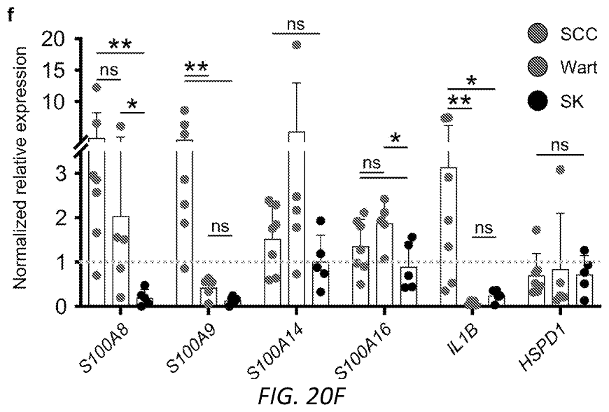

FIGS. 9A-E. DMBA-UV-induced epidermal dysplasia in uninfected SKH-1 mice is blocked in MmuPV1-colonized animals. A, Representative low and high magnification images of MmuPV1-colonized and uninfected SKH-1 skin after the completion of DMBA-UV carcinogenesis protocol are shown. Note the significant hyperplasia and dyskeratosis in uninfected SKH-1 skin, which is absent in MmuPV1-colonized skin. B, Epidermal thickness is quantified across 10 randomly selected images of the skin from SKH-1 mice in MmuPV1/DMBA-UV and −/DMBA-UV cohorts. C AND D, CD4+ T cell infiltrates in the MmuPV1-colonized and uninfected SKH-1 skin are evaluated as shown by (C) representative images of the CD4/CD3-stained skin sections and (D) quantification of CD4+ T cells per high power image of the skin. 10 random high power images of the skin from each mouse in each group are included on this graph. E, MmuPV1 PCR of skin DNA samples is used to determine MmuPV1 skin colonization at the completion of DMBA-UV treatment. Arrow points to MmuPV1 L1 PCR product (size: 339 bp). Scale bars: 100 m, error bars represent the mean+SD; ***p<0.001, ns: not significant.

FIG. 13. General binding site for RNAish and DNAish probes in human studies is shown using HPV9 genome as an example.

FIGS. 14A-B. β-HPV RNAish is validated with a positive control (wart) and quantitative real time PCR (qRT-PCR) on RNAish positive and negative human samples. A, H&E and RNAish staining of a wart from a 63-year-old immunosuppressed female are shown. Note the abundance of positive signals (red dots) throughout the wart. B, β-HPV RNAish of a skin cancer from an 87-year-old immunosuppressed female including the positive and negative control probe stains are shown. The detection of β-HPV by RNAish correlates with qRT-PCR positivity for HPV5 and 9 E6 protein transcripts in the same skin cancer. A normal skin from an 18-year-old immunocompetent African American female is stained with β-HPV RNAish probes. The lack of RNAish signal (red) in this sample correlates with undetectable HPV5, 9 or 15 E6 protein transcripts on qRT-PCR of the same sample. Scale bars: 100 m.

FIGS. 15A-C. Immunosuppressed patients have greater β-HPV viral activity in their skin lesions compared to immunocompetent patients. A, β-HPV RNAish signal counts are compared between the skin cancer cells of immunosuppressed (n=38) and immunocompetent (n=32) patients. B, A clinical image of a skin cancer surgical site shows the skin cancer (red arrow), its adjacent normal skin (green arrow) and the normal skin away from cancer site (blue arrow). C, Quantification of β-HPV RNAish signals in high power images across the immunosuppressed lesions, immunocompetent lesions and normal facial skin away from a cancer site are shown on a graph. Skin lesions include μ-HPV RNAish signal counts from skin cancer and the adjacent normal skin images (dots corresponding to cancer images are colored in maroon and dots for adjacent normal skin images are green). Thirty normal facial skin samples (blue dots) from immunocompetent patients are included in this study (18 males and 12 females, average age: 71, range: 39-94). *p<0.05, **p<0.005, ns: not significant.

FIGS. 16A-B. β-HPV viral activity is significantly increased in the basal keratinocytes of immunosuppressed patients. A, Representative low and high magnification images of β-HPV RNAish-stained normal skin samples from immunosuppressed and immunocompetent patients are shown. Note the density and size of the apparent RNAish signals in basal layer keratinocytes of an immunosuppressed patient. B, the density of β-HPV RNAish signals in basal layer keratinocytes are quantified across 38 immunosuppressed and 32 immunocompetent skin samples. Scale bar: 50 m, *p<0.05.

FIG. 17. β-HPV DNA in situ hybridization (DNAish) is used to detect μ-HPV viral load in the skin. Compared to μ-HPV RNAish that marks viral transcripts, 0-HPV DNAish is a novel tool to detect viral load at a subcellular resolution in skin keratinocytes.

FIGS. 18A-C. β-HPV viral load markedly drops in the skin cancer cells compared to their adjacent normal skin in immunocompetent patients. A, Representative DNAish of a wart, hypertrophic actinic keratosis arising in association with a wart (HAK in verruca), and SCC in immunosuppressed patients and an SCC in an immunocompetent patient are shown. B AND C, Quantification of β-HPV DNAish signals in paired samples of skin cancer and the adjacent normal skin from (B) immunosuppressed patients (n=10) and (C) immunocompetent patients (n=10) are presented as graphs. Scale bar: 100 m, *p<0.05, **p<0.01.

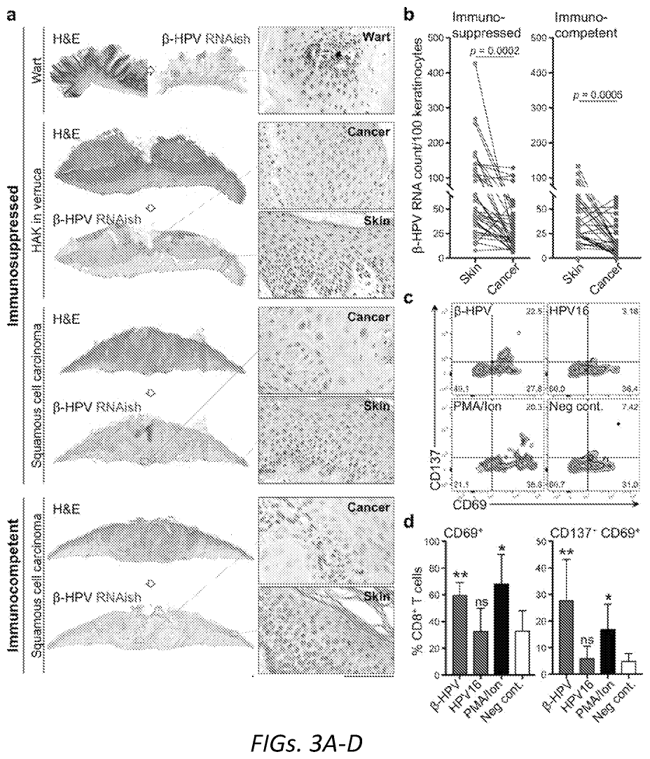

FIGS. 19A-D. Significantly fewer T and TRM cells infiltrate skin cancer and the adjacent normal skin in immunosuppressed compared to immunocompetent patients. A, Representative images of CD3/CD103-stained SCC from immunosuppressed and immunocompetent patients (the same cancers are shown for β-HPV RNA ISH and DNA ISH stains in FIG. 3A and FIG. 18A). Magnified insets highlight CD103+ TRM cells in the cancer and adjacent normal skin. Scale bars, 100 μm. B, C, CD3/CD8/CD103-stained sections of skin cancer were used to quantify tumor-infiltrating CD3+T, CD103+CD3+ TRM, CD8+T and CD103+CD8+ TRM cells infiltrating the skin cancer parenchyma (b), and CD3+T, CD103+CD3+ TRM, CD8+T and CD103+CD8+ TRM cells in the adjacent normal skin of immunosuppressed (S) versus immunocompetent (C) patients. Note that most T cells in the normal skin reside in the dermis. Stained cells were counted blindly in ten randomly selected HPF images of skin cancer and adjacent normal skin from each tissue specimen and averaged across the samples in each group; 37 immunosuppressed and 32 immunocompetent samples of skin cancer are included (skin cancer characteristics are listed in Table 3). Each dot represents the average of the T cell counts in the high-power images from each sample. Two-tailed unpaired t-test; data are mean+s.d. D, Cytotoxic degranulation of CD8+T lymphocytes after exposure to β-HPV peptides. T cells isolated from the normal facial skin of adults were exposed to β-HPV E7 peptides (far left), HPV16 E7 peptides (middle left), PMA/ionomycin (positive control; middle right) and medium (negative control; far right). Representative flow cytometry plots are shown. The percentage of CD107a+CD8+ T cells is shown on each plot. Data represent two independent sets of experiments with similar results.

FIGS. 20A-F. DAMP molecules are upregulated during the development of warts and skin cancer. A, Principle component analysis (PCA) of gene-expression profiles obtained from MmuPV1-induced warts (n=4; blue triangles), MmuPV1-infected skin (n=4; pink squares) or sham-infected skin (n=4; grey circles), and MmuPV1-infected tumors (n=4; red squares) or shaminfected tumors (n=4; black circles) of SKH-1 mice. Note that DMBA-UV induced skin tumors from MmuPV1-infected mice are indistinguishable from skin tumors from sham-infected mice, whereas both have very distinct transcriptional profiles compared with MmuPV1-driven warts. B, C, Volcano plots of differentially expressed genes in MmuPV1-versus sham-infected skin (b; n=4 per group), and skin tumors and warts (n=12) versus MmuPV1- and sham-infected skin (c; n=8). Gm5416 is also known as Csta3. P values were calculated using the DESeq2 R package (v.2 1.6.3), and the resulting P values were adjusted using the Benjamini-Hochberg method for controlling the false discovery rate. The 20 genes that were upregulated in skin tumors and warts compared with MmuPV1- and sham-infected skin are shown in the table on the left. D-F, Analysis of the expression of immune genes in human skin lesions on the basis of the mouse RNA-seq data. D, Representative macroscopic and H&E stained histological images of SCC, wart, seborrheic keratosis (SK) and unaffected human skin. Scale bar, 500 m. E, Relative gene expression in SCCs (n=7) and warts (n=5) compared with normal skin (n=8). F, Normalized relative gene expression in SCCs (n=7), warts (n=5) and seborrheic keratosis (n=5) compared for several DAMP genes. Average relative gene expression in the normal skin was used for normalization. GAPDH is used as the reference gene. Two-tailed Mann-Whitney U test; *P<0.05, **P<0.01, NS, not significant; data are mean+s.d. (e, f).

### DETAILED DESCRIPTION

Human papillomaviruses (HPV), particularly of the low-risk beta (0) genus, have been found in more than 80% of SCCs among OTRs.²⁻⁵ Therefore, a potential viral cause of skin cancer has been proposed.¹⁴ β-HPVs are a cause of benign cutaneous warts and, together with other cutaneotropic low-risk HPV genera, are ubiquitously present on the skin of immunocompetent adults as normal flora.^{3,4,15,16} In contrast to high-risk α-HPV, there are no predominant μ-HPV subtypes identified in skin cancers¹⁴ and the β-HPV genome is rarely integrated into the DNA of cancer cells.⁵ Additionally, transcriptome analysis has failed to identify papillomavirus gene expression in SCCs of immunocompetent or immunosuppressed patients.⁴ In skin cancers positive for β-HPV, the viral load in tumor cells is less than one copy per cell.¹⁴ Furthermore, the prevalence of β-HPV DNA in actinic keratosis (SCC precursor lesion) is higher than in SCC in immunocompetent patients and HPV is mostly present in superficial layers, not basal proliferative regions of skin cancers.^{17,18} These findings contributed to the “hit-and-run” theory to explain HPV's role in skin carcinogenesis, in which the virus facilitates the initiation of a skin cancer but is later lost during tumor maintenance.^{5,19}

The findings reported herein reveal a novel role for commensal HPVs in the development of nonmelanoma skin cancers. The clinical study demonstrates that the immunosuppression has no impact on the anatomical distribution of skin cancer, which is tightly associated with the areas of greatest sun damage. The distinct localization of skin cancers away from the intermittently sun-damaged and sun-protected skin, which contain the majority of HPV-driven warts, suggests that commensal HPVs do not initiate skin cancer alone or in combination with UV in immunosuppressed patients. The MmuPV1 colonization model enabled mechanistic studies of the relationship between papillomavirus and skin cancer in the context of an intact immune system. Using this model, we show that MmuPV1-colonized immunocompetent mice are protected against chemical and UV-induced skin cancer compared to their uninfected counterparts. Further, we demonstrate that T cell immunity against MmuPV1 renders the MmuPV1-colonized mice protected against carcinogen-induced skin tumors. Finally, our innovative approach to viral RNA and DNA detection on histological sections with a subcellular resolution reveals a negative selection against β-HPV viral activity and load in malignant keratinocytes as they evade the antiviral T cell immunity in the skin to form cancer.

The present findings support a novel explanation for the role of low-risk commensal HPVs in skin cancer development. The extremely low prevalence of warts in immunocompetent adults²⁴ highlights the ability of a functional immune system to target and eliminate HPV-infected proliferating cells. Likewise, anti-HIPV immunity halts skin cancer development due to recognition of commensal HPVs in the premalignant cells, which shares the antigenic/immunogenic properties of a wart and is effectively eliminated. This protective immunity is compromised in immunosuppressed patients leading to markedly increased skin cancers, warts and HPV viral load in this population. Therefore, the increased skin cancer risk upon immunosuppression represents the loss of the protective effect of antiviral immunity, rather than the gain of susceptibility to a HPV-driven skin cancer.

The disparate anatomical distribution of skin cancers and warts points to UV exposure as the dominant determinant of skin cancer risk in immunosuppressed patients. This finding is supported by SCC transcriptome analysis, which has demonstrated an indistinguishable pattern of mutated genes in SCCs of immunosuppressed and immunocompetent patients.²⁵ If commensal HPVs exerted a significant mutagenic contribution to SCC development, immunosuppressed patients would be expected to have fewer UV-induced mutations and/or a distinct pattern of mutated genes. This is in stark contrast to high-risk oncogenic viruses like Merkel cell polyomavirus (MCPyV) in the skin, which causes Merkel cell carcinoma with low mutational burden compared to highly mutated MCPyV-negative Merkel cell carcinoma caused by UV.^{26,27} These observations together with low HPV load and its lack of transcriptional activity in skin cancers^{4,14} provide ample evidence that commensal HPVs' contribution to skin cancer development is negligible.

The experimental studies on MmuPV1 in Wt mice demonstrates the protective role of commensal papillomavirus against skin cancer in immunocompetent hosts. Suppression of MmuPV1-induced warts has been shown to be T cell-mediated.^{22,28} Here, we show that virus-specific T cells are sufficient to render MmuPV1-colonized mice protected against carcinogen-induced skin cancer. Interestingly, MmuPV1-colonized SKH-1 mice were also protected from UV-induced epidermal dysplasia, which may suggest a role for commensal HPVs in maintaining the homeostatic state of highly mutated sun-damaged human skin.²⁹ Previous studies on animal models of HPV skin infection have implicated HPV as a driver of skin cancer. The cell-autonomous proliferative effect of commensal HPVs in keratinocytes as wart-causing agents is evident.³⁰ However, the use of animals with transgenic expression of viral E6/7 proteins in isolation,³¹ immunodeficient mice³⁰ or the immunosuppressive doses of UV^{23,32,33} has hindered published work from fully interrogating the role of low-risk commensal papillomaviruses in skin carcinogenesis due to the lack of a physiologic immune response. These studies highlight the importance of the use of a fully infectious virus and the antiviral immune response in order to reach translational conclusions about the role of virome in human disease.

Evidence of CD8⁺ T cells responsive to β-HPV peptides in the normal adult skin supports a role for the HPV-specific adaptive immune responses against skin's normal flora. Without wishing to be bound by theory, it is believed that together with the loss of β-HPV activity and viral load in skin cancers compared to the normal skin, commensal β-HPVs function as immunogenic tags in abnormally dividing keratinocytes. They engage a cytotoxic T cell response against any proliferative lesions generated by the cells that contain the active virus. Therefore, T cell-based vaccines against β-HPVs can provide an innovative approach to boost the antiviral immunity in the skin and help prevent warts and skin cancers in high-risk populations, especially OTRs prior to transplantation. Current B cell-based HPV vaccines block the infection of epithelial cells with high-risk HPVs of alpha genus.³⁴ In contrast, the goal of a β-HPV vaccine will be to capitalize on the beneficial effect of β-HPV colonization by potentiating the cell-mediated antiviral immunity in the colonized skin in order to prevent wart and skin cancer development. Unlike peptides from 3-HPVs that are normal skin flora,^{3,4,15,16} high-risk HPV16 peptides did not elicit any response from skin-resident T cells. This highlights the importance of the skin commensal HPVs in orchestrating an anti-tumor immune response and the need to boost this immunity for skin cancer prevention and treatment. Based on the present data, we now understand commensal viruses to be agents that can help our immune system protect against cancer development, even in immunocompetent populations.

In summary, we demonstrate a novel and beneficial effect of commensal HPVs in protecting the skin against cancer by alerting the cytotoxic immunity against any proliferative lesion in the skin. Considering the emerging diversity of virome in the skin,³⁵ it is critical to identify the composition of the skin-resident viral communities in immunocompetent and immunosuppressed individuals and determine how these viruses contribute to human health and disease.

T Cell-Based Vaccines Against β-HPVs

Current vaccines against cancer-causing viruses like high-risk alpha-type human papilloma viruses (α-HPV) are designed to activate B cells leading to antiviral antibody production that prevents the viral infection of the target tissues (e.g., cervix) in the first place. However, we have recently discovered that commensal viruses, which colonize the target tissue shortly after birth in all individuals (e.g., low-risk beta-type HPVs colonization of skin) play a protective role against carcinogen-driven cancers (e.g., skin cancer) by inducing an antiviral T cell immunity capable of eliminating any proliferative lesion containing the HPV virus. In other words, in adult individuals who are immune to warts (HPV-driven lesion on the skin), HPV infection functions as a tag on the skin cells, which alerts the T cells as soon as the cell starts to proliferate abnormally either in the context of HPV-driven wart or carcinogen-driven skin cancer. Therefore, boosting the T cells immunity by a T cell-directed vaccine will heighten the protective impact of the commensal viruses without eliminating their dormant colonization of the target tissues. Described herein are compositions that can be used to induce a T cell-based immune response against β-HPVs, thereby reducing the risk that the subject will develop skin cancer. The vaccines induce T cell immunity against commensal viruses that have already infected the tissue, with the goal not to prevent or eliminate the infection but rather to use of the virus presence in all cells to boost the detection of early cancerous clones and their elimination by T cells. Current high-risk HPV vaccines for cervical and head and neck cancer prevention are meant to prevent infection in the first place and have minimal efficacy in individuals already infected with the virus.

### Antigen Peptides

In some embodiments, the present compositions include a plurality of antigenic peptides derived from (i.e., comprising a fragment of, i.e., consecutive amino acids from) proteins, e.g., E1, E2, E6, or E7 proteins, from commensal human papilloma viruses, e.g., low risk cutaneotropic α-HPV, β-TPV, 7-HIPV and/or i-HPV strains such as those listed in Table A. In some embodiments, the compositions do not include peptides derived from HPV types that are associated with cancer, e.g., high-risk HIPVs such as HIPV16 or 18. See, e.g., Ma et al., J Virol. 2014 May; 88(9): 4786-4797; Doorbar et al., Rev Med Virol. 2015 March; 25(Suppl Suppl 1): 2-23; Doorbar et al., The biology and life-cycle of human papillomaviruses. Vaccine 2012; 30(Suppl 5): F55-F70; de Villiers, Virology 2013; 445(1-2): 2-10; and U.S. Pat. No. 8,652,482, which are incorporated herein by reference. Other commensal HPV types that are not “high-risk” V-HPVs can be used; Table A is an exemplary but not exhaustive list.

TABLE A

| HPV | HPV | HPV | | | | | | |
|---|---|---|---|---|---|---|---|---|
| Genus | Species | Type | E1 | E2 | E4 | E5 | E6 | E7 |
| α | 4 | HPV2 | ABO14922.1 | ABO14923.1 | ABO14924.1 | — | ABO14920.1 | ABO14921.1 |
| α | 2 | HPV3 | CAA52471.1 | CAA52472.1 | — | — | CAA52469.1 | — |
| α | 8 | HPV7 | CAA52478.1 | CAA52479.1 | — | — | NP_041854.1 | CAA52477.1 |
| α | 2 | HPV10 | CAA52491.1 | CAA52492.1 | — | — | CAA52489.1 | CAA52490.1 |
| α | 4 | HPV27 | CAA52538.1 | CAA52539.1 | BAE16267.1 | — | CAA52536.1 | CAA52537.1 |
| α | 2 | HPV28 | AAA79424.1 | AAA79425.1 | P51896.2 | — | AAA79422.1 | AAA79423.1 |
| α | 4 | HPV57 | CAA39432.1 | CAA39433.1 | CAA39434.1 | — | CAA39430.1 | CAA39431.1 |
| α | 2 | HPV77 | CAA75465.1 | CAA75466.1 | CAA75469.1 | — | CAA75463.1 | CAA75464.1 |
| β | 1 | HPV5 | AFL02855.1 | AFL02856.1 | AAA46987.1 | AAA46988.1 | CAA52693.1 | CAA52694.1 |
| β | 1 | HPV8 | P06420.1 | P06422.1 | P06425.2 | — | P06428.1 | P06430.1 |
| β | 2 | HPV9 | CAA52485.1 | CAA52486.1 | — | — | CAA52483.1 | CAA52484.1 |
| β | 1 | HPV12 | CAA52498.1 | CAA52499.1 | — | — | CAA52496.1 | CAA52497.1 |
| β | 1 | HPV14 | CAA52502.1 | CAA52503.1 | S65692 | — | P28830.1 | BAA09114.1 |
| β | 2 | HPV15 | CAA52508.1 | CAA52509.1 | — | — | CAA52506.1 | CAA52507.1 |
| β | 2 | HPV17 | CAA52514.1 | CAA52515.1 | AFL02863.1 | — | CAA52512.1 | CAA52513.1 |
| β | 1 | HPV19 | CAA52520.1 | CAA52521.1 | — | — | P36806.1 | CAA52519.1 |
| β | 1 | HPV20 | ARV85572.1 | ARV85573.1 | ARV85574.1 | — | ARV85578.1 | ARV85575.1 |
| β | 1 | HPV21 | AAA79396.1 | AAA79397.1 | AAA79398.1 | — | AAA79394.1 | AAA79395.1 |
| β | 2 | HPV22 | AAA79403.1 | AAA79404.1 | AAA79405.1 | — | AAA79401.1 | AAA79402.1 |
| β | 2 | HPV23 | AAA79410.1 | AAA79411.1 | AAA79412.1 | — | AAA79408.1 | AAA79409.1 |
| β | 1 | HPV24 | AAA79417.1 | AAA79418.1 | AAA79419.1 | — | AAA79415.1 | AAA79416.1 |
| β | 1 | HPV25 | CAA52526.1 | CAA52527.1 | — | — | CAA52524.1 | CAA52525.1 |
| β | 1 | HPV36 | AAA79438.1 | AAA79439.1 | AAA79440.1 | — | AAA79436.1 | AAA79437.1 |
| β | 2 | HPV37 | AAA79445.1 | AAA79446.1 | AAA79447.1 | — | AAA79443.1 | AAA79444.1 |
| β | 2 | HPV38 | AAA79452.1 | AAA79453.1 | AAA79454.1 | — | AAA79450.1 | AAA79451.1 |
| β | 1 | HPV47 | AAA46978.1 | AAA46979.1 | AAA46980.1 | — | AAA46976.1 | AAA46977.1 |
| β | 3 | HPV49 | CAA52581.1 | CAA52582.1 | — | — | CAA52579.1 | CAA52580.1 |
| β | 3 | HPV75 | CAA75451.1 | CAA75452.1 | CAA75455.1 | — | CAA75449.1 | CAA75450.1 |
| β | 3 | HPV76 | CAA75458.1 | CAA75459.1 | CAA75462.1 | — | CAA75456.1 | CAA75457.1 |
| β | 2 | HPV80 | CAA75472.1 | CAA75473.1 | CAA75474.1 | — | CAA75470.1 | CAA75471.1 |
| β | 4 | HPV92 | NP_775307.1 | NP_775308.1 | NP_775309.1 | — | NP_775305.1 | NP_775306.1 |
| β | 1 | HPV93 | AAQ88287.1 | AAQ88283.1 | AAQ88288.1 | — | AAQ88286.1 | AAQ88282.1 |
| β | 5 | HPV96 | NP_932321.1 | NP_932322.1 | NP_932323.1 | — | NP_932319.1 | NP_932320.1 |
| β | 2 | HPV107 | ABN79869.1 | ABN79870.1 | ABN79871.1 | — | ABN79867.1 | ABN79868.1 |
| β | 2 | HPV110 | ACC78265.1 | ACC78267.1 | ACC78266.1 | — | ACC78263.1 | ACC78264.1 |
| β | 2 | HPV111 | ACC78272.1 | ACC78274.1 | ACC78273.1 | — | ACC78270.1 | ACC78271.1 |
| β | 1 | HPV129 | ADR77929.1 | ADR77930.1 | ADR77931.1 | — | ADR77932.1 | ADR77933.1 |
| β | 1 | HPV151 | CBK38955.1 | CBK38956.1 | CBK38957.1 | — | CBK38953.1 | CBK38954.1 |
| β | 2 | HPV164 | AFV27105.1 | AFV27106.1 | AFV27107.1 | — | AFV27103.1 | AFV27104.1 |
| γ | 1 | HPV4 | CAA50159.1 | CAA50160.1 | CAA50161.1 | — | CAA50157.1 | CAA50158.1 |
| γ | 22 | HPV30 | ALT54645.1 | ALJ32745.1 | ALJ32746.1 | — | ALT54643.1 | ALT54644.1 |
| γ | — | HPV33 | ACL12328.1 | ACL12329.1 | ACL12330.1 | ACL12331.1 | AGM34423.1 | AGM34459.1 |
| γ | 22 | HPV35 | ARQ82604.1 | ARQ82605.1 | ARQ82607.1 | ARQ82606.1 | ASA39832.1 | ASA39833.1 |
| γ | 3 | HPV48 | NP_043418.1 | NP_043419.1 | NP_043420.1 | — | NP_043416.1 | NP_043417.1 |
| γ | 2 | HPV50 | NP_043425.1 | NP_043426.1 | NP_043427.1 | — | NP_043423.1 | NP_043424.1 |
| γ | 4 | HPV60 | NP_043439.1 | NP_043440.1 | NP_043441.1 | — | NP_043437.1 | NP_043438.1 |
| γ | 1 | HPV65 | CAA50173.1 | CAA50174.1 | CAA50175.1 | — | CAA50171.1 | CAA50172.1 |
| γ | 5 | HPV88 | ABR20504.1 | ABR20505.1 | ABR20506.1 | — | ABR20502.1 | ABR20503.1 |
| γ | 1 | HPV95 | CAF05704.1 | CAF05705.1 | CAF05706.1 | — | CAF05702.1 | CAF05703.1 |
| γ | — | HPV101 | AAZ39508.1 | AAZ39509.1 | AAZ39510.1 | — | — | AAZ39507.1 |
| γ | 14 | HPV103 | AAZ39486.1 | AAZ39487.1 | AAZ39488.1 | — | — | AAZ39485.1 |
| γ | 9 | HPV121 | ADH29807.1 | ADH29808.1 | ADH29809.1 | — | ADH29805.1 | ADH29806.1 |
| γ | 15 | HPV135 | AEM24595.1 | AEM24596.1 | AEM24597.1 | — | AEM24593.1 | AEM24594.1 |
| γ | — | HPV161 | AFV27124.1 | AFV27125.1 | — | — | AFV27122.1 | AFV27123.1 |
| γ | 8 | HPV164 | AFV27105.1 | AFV27106.1 | AFV27107.1 | — | AFV27103.1 | AFV27104.1 |
| γ | 16 | HPV186 | ANG08953.1 | ANG08954.1 | — | — | ANG08951.1 | ANG08952.1 |
| γ | 2 | HPV200 | AKP16336.1 | AKP16337.1 | — | — | AKP16334.1 | AKP16335.1 |
| μ | 1 | HPV1 | ? | ? | ? | ? | ? | ? |
| μ | — | HPV63 | CAA50166.1 | CAA50167.1 | CAA50168.1 | — | CAA50164.1 | CAA50165.1 |

The peptides can be derived from any antigenic protein in the virus; in some embodiments, the peptides are derived from an E1, E2, E4, E5, E6 or E7 protein. Sequences for these proteins in a number of commensal strains are provided. In some embodiments, at least 50 or more, 100, 150, 200, 250, 300, 350, 400, 450, 500, or more different peptides (i.e., peptides having different sequences) are included in the compositions. In some embodiments, at least 50 or more, 100, 150, 200, 250, 300, 350, 400, 450, 500, or more different peptides from each virus strain are included in the compositions, and peptide from two or more virus strains are included.

In some embodiments, the peptides are of a length that is optimized for MHCI/MHCII presentation, e.g., 9-30 amino acids, e.g., 12-25, 12-18, 12-16, 13-16, 14-16, or 15 amino acids. The sequences of the peptides can be synthetic long overlapping peptides, e.g., identified, e.g., bioinformatically to predict antigenicity and/or generated using a moving window of overlapping peptides to cover the entire protein, e.g., 15 amino acid peptides with 10 amino acid overlap (similar to the “gene walk” methods used to identify optimal antisense oligonucleotides). In some embodiments, overlapping synthetic long peptides (SLPs) are used (Zom et al., Cancer Immunol Res. 2014 August; 2(8):756-64). The compositions can include a plurality of peptides derived from one or more (e.g., a plurality of) different virus strains. The peptides are preferably synthetic peptides; methods for synthesizing peptides are known in the art, including solution-phase techniques and solid-phase peptide synthesis (SPPS). See, e.g., Petrou and Sarigiannis, Ch. 1—Peptide synthesis: Methods, trends, and challenges, In: Editor(s): Sotirios Koutsopoulos, Peptide Applications in Biomedicine, Biotechnology and Bioengineering, Woodhead Publishing, 2018, pages 1-21; and Chandrudu et al., Molecules 2013, 18, 4373-4388.

### Antigenic Proteins

In some embodiments, the present compositions can include a plurality of proteins, e.g., virus-like particles containing of E1, E2, E6, or E7 proteins from commensal human papilloma viruses, e.g., low risk α-HPV, β-HPV, γ-HPV and/or β-HPV strains such as those listed in Table A (see, e.g., Yang et al., Virus Res 231, 148-165 (2017); Hancock et al., Therapeutic HPV vaccines. Best Pract Res Clin Obstet Gynaecol 47, 59-72 (February 2018); Joh et al., Exp Mol Pathol.; 93(3):416-21 (2012)).

### Nucleic Acid-Based Vaccines

In some embodiments, the present compositions can include a plurality of DNA plasmids and/or RNA replicons that contain nucleotide sequences to express proteins or antigenic peptides derived from (i.e., comprising a fragment of, i.e., consecutive amino acids from) proteins, e.g., E1, E2, E6, or E7 proteins, from commensal human papilloma viruses, e.g., low risk α-HPV, β-HPV, γ-HPV and/or μ-HPV strains such as those listed in Table A (see, e.g., Yang et al., Virus Res 231, 148-165 (2017); Hancock et al., Therapeutic HPV vaccines. Best Pract Res Clin Obstet Gynaecol 47, 59-72 (2018)).

### Live Vector-Based Vaccines

In some embodiments, the present compositions can include a plurality of viral vectors that are engineered to express proteins or antigenic peptides derived from (i.e., comprising a fragment of, i.e., consecutive amino acids from) proteins, e.g., E1, E2, E6, or E7 proteins, from commensal human papilloma viruses, e.g., low risk α-HPV, β-HPV, γ-HPV and/or μ-HPV strains such as those listed in Table A (see, e.g., Yang et al., Virus Res 231, 148-165 (2017); Hancock et al., Therapeutic HPV vaccines. Best Pract Res Clin Obstet Gynaecol 47, 59-72 (2018)).

Viral vectors for use in the present methods and compositions include recombinant retroviruses, adenovirus, adeno-associated virus, alphavirus, and lentivirus.

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in numerous tissues including the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficient and selectivity, e.g., biotinylated AAV vectors, directed molecular evolution, self-complementary AAV genomes and so on. In some embodiments, AAV1 is used.

Alternatively, retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Alphaviruses can also be used. Alphaviruses are enveloped single stranded RNA viruses that have a broad host range, and when used in gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1(1): 13-25; Lundstrom, Viruses. 2014 June; 6(6): 2392-2415; Lundstrom, Curr Gene Ther. 2001 May; 1(1):19-29; Rayner et al., Rev Med Virol. 2002 September-October; 12(5):279-96.

### Live Commensal HPV Vaccine Strategy

A live commensal HPV vaccine strategy can be used to optimally boost antiviral T cell immunity in the skin to prevent cancer development and treat early SCCs with active virus, which include actinic keratosis, SCC in situ and early invasive SCC. Described herein is a platform to generate and expand live low-risk HPVs in culture in order to generate live and live attenuated HPV vaccine for use in patients.

The present prophylactic cancer vaccine takes advantage of the skin's widespread colonization with “good” viruses in order to prevent and treat skin cancer. The only T cell-based vaccine strategy with proven efficacy is a live-attenuated varicella zoster virus vaccine to prevent shingles: Zostavax (Sullivan et al., Current opinion in immunology. 2019; 59:25-30. Epub 2019/04/11). In the case of Zostavax, the targeted virus, varicella zoster, is the cause of chicken pox and shingles; thus, the attenuated virus had to be developed for the safety of the vaccination. A T cell-based vaccine strategy against commensal HPVs targets low-risk papillomaviruses that are normal flora of humans. As such, there is no evidence of these viruses causing any serious illness in adult individuals besides benign skin warts, and only in highly immunosuppressed patients. Therefore, a live commensal papillomavirus vaccine is an ideal platform for skin cancer prevention as it can efficiently infect the cells and the viral antigenic peptides can be effectively presented to T cells in major histocompatibility complex (MHC) while avoiding neutralizing antibodies.

Live HPV Vaccines:

An in vitro culture system can be used to expand cutaneotropic HPVs. Commensal HPVs can be obtained using known methods, e.g., isolated from warts of adult immunosuppressed patients. Next, the purified virus (Kreider et al., Virology. 1990; 177(1):415-7) is transferred to an organotypic raft culture model using Human Primary Keratinocytes (low passage Human Foreskin Keratinocytes (HFKs) rather than immortalized cell lines (Bienkowska-Haba et al., PLoS Pathog. 2018; 14(3):e1006846. Epub 2018/03/02; Ozbun et al., Curr Protoc Microbiol. 2014; 34:14B 3 1-8. Epub 2014/08/02; Anacker et al., Journal of visualized experiments: JoVE. 2012(60). Epub 2012/03/08)). The difficulty in transfecting HPV genome into keratinocytes is resolved by using the extracellular matrix (ECM)-to-cell infection method as HPVs preferentially bind in vivo and in vitro to the basement membrane and the ECM secreted by keratinocytes (Richards et al., Viruses. 2014; 6(12):4856-79). This involves the seeding of the cells on the surface of a collagen gel and later transferring this gel onto a stainless-steel grid with culture medium as to create an air-to-medium interface. The possibility to expand commensal HPVs in culture enables the use of live commensal HPVs in our prophylactic cancer vaccine.

Live Attenuated HPV Vaccines:

Live attenuated HPV vaccines can also be used. The low-risk commensal HPV E6 protein has been shown to interfere with Notch signaling, which drives keratinocyte differentiation and cell cycle arrest (Tan et al., Proceedings of the National Academy of Sciences of the United States of America. 2012; 109(23):E1473-80. Epub 2012/05/024). Specifically, E6 binds to the C-terminal domain of Mastermind-like (MAML1) protein, a member of the Notch transcription complex (Id.). This allows for suppression of keratinocyte differentiation and maintains an advantageous cellular environment for low-risk HPV replication, leading to wart development. Consequently, the mutation of the gene encoding for the E6 protein of commensal HPVs at its binding site to the LXXLL domain of MAML-1 allows for the development of a live attenuated virus that is safe for use in vaccine. The protein E6 contains four zinc-binding domains, each harboring two C-x-x-C motifs (Nomine et al., Mol Cell. 2006; 21(5):665-78. Epub 2006/03/02). Specifically, the N-terminal domain has been suggested to be the binding site of E6 proteins (Id.). In some embodiments, the virus includes one or more mutations of C-x-x-C motifs to S-x-x-S motifs in the amino-terminal domain of the E6 protein, to prevent binding to MAML-1 and inhibit wart development upon infection with the mutated virus in human tissue. In fact, these specific cysteine to serine mutations inhibit binding of zinc ions to the zinc-binding domains, thereby hindering the protein's binding abilities.

It has also been shown that HPVs bind the LXXLL consensus sequence of target proteins like MAML-1 (Tungteakkhun et al., Arch Virol. 2008; 153(3):397-408. Epub 2008/01/04). In some embodiments, the virus includes mutations in an LXXLL-binding motif (see, e.g., Brimer et al., PLoS Pathog. 2017 December; 13(12): e1006781), e.g., in an amino-terminal E6 zinc-binding domain and the carboxy-terminal zinc-binding domain (Vande Pol and Klingelhutz, Virology, 2013, 445(1-2):115-137), or in one or more of 8S9Δ10T, I128T, or A146-151 (White et al., J Virol. 2012 December; 86(24): 13174-13186).

The commensal HPV clinical isolates will be attenuated as above so that they are able to complete their full life cycle, without retaining their pathogenic ability to cause wart development. Before introducing HPV clinical isolates into the in vitro culture system, attenuated mutants are generated using oligonucleotide-directed site-specific mutagenesis. Oligonucleotides harboring a desired mutation will be introduced into the HPV genome cloned into a plasmid or a bacterial artificial chromosome (BAC), a method that has been previously described and yielded infectious virions using the organotypic raft culture model (Meyers et al., Journal of virology. 2002; 76(10):4723-33. Epub 2002/04/23). Recombinant viral genome is introduced into Human Primary Keratinocytes. After transfection, the cells are differentiated and grown using the organotypic culture model, which supports the full HPV life cycle.

### T Cell Adjuvant

The compositions can also include an adjuvant to increase T cell response. For example, nanoparticles that enhance T cell response can be included, e.g., as described in Stano et al., Vaccine (2012) 30:7541-6 and Swaminathan et al., Vaccine (2016) 34:110-9. See also Panagioti et al., Front. Immunol., 16 Feb. 2018; doi.org/10.3389/fimmu.2018.00276. Alternatively or in addition, an adjuvant comprising poly-ICLC (carboxymethylcellulose, polyinosinic-polycytidylic acid, and poly-L-lysine double-stranded RNA), Imiquimod, Resiquimod (R-848), CpG oligodeoxynuceotides and formulations (IC31, QB10), AS04 (aluminium salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant) can also be used. In some embodiments, topical imiquimod and/or topical 5-fluorouracil and/or topical calcipotriene (calcipotriol) in combination with 5-fluorouracil (e.g., as described in Cunningham et al., J Clin Invest. 2017; 127(1):106-116) could serve as adjuvants for the vaccine (this would be particularly applicable in subjects with pre-malignant skin lesions, who are commonly treated with these topical agents). See, e.g., Khong and Willem, Journal for ImmunoTherapy of Cancer 4:56 (2016); Coffman et al., Immunity. 2010 Oct. 29; 33(4): 492-503; Martins et al., EBioMedicine 3:67-78, 2016; and Del Giudice, Seminars in Immunology, 2018, doi.org/10.1016/j.smim.2018.05.001.

### Compositions

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, intratumoral, intramuscular or subcutaneous administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, intramuscular, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Subjects

The vaccine compositions described herein can be used to boost immunity against skin cancer in immunocompetent subjects, as well as immunosuppressed or immunocompromised patients who have reduced T cell immunity against β-HPVs and are prone to developing multiple skin warts and cancers (loaded with virus) with poor prognosis. In some embodiments, the subjects do not have cancer (e.g., do not have skin cancer). In some embodiments, the subjects are at high risk (i.e., have a risk that is above that of the general population) of developing skin cancer, e.g., non-melanoma, e.g., squamous cell carcinoma of the skin. For example, the subject may have a family history of skin cancer, a personal history of excessive sun exposure/sunburns, fair skin, residence in sunny or high-altitude climates, exposure to radiation or carcinogenic substances such as arsenic, moles, precancerous skin lesions, or a family history or personal history of skin cancer.

In some embodiments, the subject may be immunosuppressed, e.g., due to an organ transplant, an acquired immunodeficiency, e.g., HIV/AIDS, or primary human immunodeficiency. In some embodiments, the subject is immunosuppressed due to aging. The present methods and compositions are helpful in aging individuals, as aging is associated with immunosenescence; thus, even those who are aging normally would benefit from vaccine to boost their antiviral immunity. Thus in some embodiments, the subject is aging, e.g., is at least 50, 55, 60, 65, 70 75, 80, 85, or 90 years old.

Subjects who can be treated using the present methods include mammals, e.g., human and non-human veterinary subjects.

Methods of Inducing Anti-Cancer Immunity

The present compositions can be used to induce anti-cancer immunity, to reduce the risk of developing skin cancer, e.g., non-melanoma, e.g., squamous cell carcinoma of the skin. The methods include administering one or more doses of the vaccine compositions described herein to a subject, e.g., a subject in need thereof.

The compositions are administered in an effective amount. An “effective amount” is an amount sufficient to effect beneficial or desired results. For example, an effective amount is one that achieves a desired therapeutic effect, e.g., an amount necessary to treat a disease, or to reduce risk of development of disease or disease symptoms (also referred to as a therapeutically effective amount or a prophylactically effective amount, respectively). An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, the methods can include administering a first dose, followed by a second dose at a later time (e.g., a “booster” dose), e.g., at 1, 2, 4, 6, 8, 12, 18, 24, or 52 weeks later.

Dosage, toxicity and therapeutic efficacy of the therapeutic compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit high therapeutic indices are preferred. While compositions that exhibit toxic side effects may be used, care should be taken to minimize and reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compositions used in the methods described herein, the therapeutically effective dose can be estimated initially from cell culture assays. A dose may be formulated in animal models. Such information can be used to more accurately determine useful doses in humans.

The methods can also include administration of one or more other treatments known in the art for skin cancer, e.g., in subjects who have skin cancer, or treatment to reduce the risk of developing skin cancer. For example, a combination treatment with the compositions described herein plus field treatments for actinic keratosis (to reduce risk of developing skin cancer), e.g., topical 5-fluorouracil, topical imiquimod, topical calcipotriene plus 5-fluorouracil, Ingenol mebutate, and photodynamic therapy. In some embodiments, these agents boost antigen presentation (innate signals) while the present compositions boost antigen recognition by T cells. In subjects who have skin cancer, surgical treatments (e.g., Mohs surgery, excisional surgery, curettage and electrodessication (electrosurgery), cryosurgery, or laser surgery); radiation therapy; photodynamic therapy; topical medications (e.g., topical 5-fluorouracil, topical imiquimod, topical calcipotriene plus 5-fluorouracil, or Ingenol mebutate); or systemic medications (e.g., cemiplimab-rwlc, e.g., for subjects with metastatic squamous cell carcinoma of the skin), can be used in combination with the present methods.

### EXAMPLES

The invention is further described in the following examples, which do not limit the scope of the invention described in the claims.

Methods

The following materials and methods were used in the Example below.

Human Studies

The participants in the clinical study were consented for the review of their medical records, access to their archived skin cancers and contribution of wart biopsy samples to the study in the High Risk Skin Cancer Clinics at Massachusetts General Hospital. Discarded de-identified normal human skin samples were obtained through Mohs surgery clinics at Massachusetts General Hospital. The skin lesions and normal skin samples were (a) processed for immune cell or RNA isolation and (b) fixed in formalin and embedded in paraffin for histological assays.

Animal Studies

All mice were housed under pathogen-free conditions in the animal facilities at Massachusetts General Hospital and University of Louisville in accordance with animal care regulations. 6-8 weeks old female C57BL/6J (The Jackson Laboratory, Bar Harbor, Me., strain code: 000664), FVB (Charles River, Wilmington, Mass., strain code: 207), and SKH-1 Elite (Charles River, strain code: 477) were used in the immunocompetent arms of this study. CD4^{-/-}; CD8^{-/-} mice in FVB background were used as T cell deficient hosts (provided by Dr. David G. DeNardo; CD8^{-/-}: The Jackson Laboratory, strain code: 032563). MmuPV1-infected mice were housed in a biocontainment unit in an animal facility at University of Louisville in accordance with animal care regulations.

Statistical Analysis

Two-tailed fisher's exact test was used as the test of significance for skin cancer and wart anatomical distribution outcomes. Pearson's χ² tests were used for other categorical variables. Two-tailed Mann-Whitney U test was used for tumor counts and T cell activation assay. Two-tailed paired t-test was used for comparing RNAish and DNAish signal counts between skin cancers and their adjacent normal skin. Two-tailed unpaired t-test was used for epidermal thickness, immunostained T cell counts, RNAish signal counts comparing skin lesions to normal human skin, and other continuous variables. Log-rank test was used as the test of significance for time to tumor onset outcomes. A P value less than 0.05 was considered significant. All the bar graphs show mean+standard deviation.

MmuPV1 Inoculation

MmuPV1 viral stock was prepared from MmuPV1-induced muzzle papillomas of B6.Cg-Foxn1^{1nu}/Foxn1^{1nu} mice following a protocol described previously.³⁶ Back skin of the Wt and CD4^{-/-}; CD8^{-/-} mice was shaved with electric razor and waxed. Next, skin was scarified using a nail file×10-20 passages across the skin to generate microaberrations in the skin barrier, which was accompanied by skin erythema. 20 μl of virus inoculum was pipetted onto scarified skin and spread homogenously. The same viral inoculum was used for all infected mice, which yielded confluent wart development on the back skin of T cell-deficient FVB mice. Sham-infected mice received 20 μl of sterile normal saline. Vaseline gauze (McKesson, San Francisco, Calif., catalog no. 61-20056) was cut to fit the site of the injury and applied under a standard bandaid. 0.5 mg/kg of meloxicam (Boehringer Ingelheim Vetmedica, St. Joseph, Mo.) was injected subcutaneously for pain relief and again the next day. Bandaids were removed at 48 hr and 200 μl of sterile normal saline was injected subcutaneously to any lethargic mice.

PCR Detection of NMmuPV1 in Mouse Skin

To confirm skin colonization after MmuPV1 back skin infection and at the completion of carcinogenesis protocols, DNA were isolated from the skin biopsies using DNeasy Blood & Tissue Kit (Qiagen, Hilden, Germany, catalog no. 69506). PCR amplification of MmuPV1 L1 protein was performed following previously described method (primers are listed in the following Table).¹

| | | SEQ ID |
|---|---|---|
| Gene Name | | NO: |
| PCR: | | |
| MmuPV1 L1 | forward: GAGCTCTTTGTTACTGTTGTC | 1. |
| | reverse: ATCCTCTCTTTCCTTGGGC | 2. |
| qRT-PCR: | | |
| HPV5 E6 | forward: GCCGAACACCAACAGAAACT | 3. |
| | reverse: AACAATCAATCACAGGGATGC | 4. |
| HPV9 E6 | forward: AGCTTATTTGGACAGAGGAGGA | 5. |
| | reverse: GTGCAGACGCATAAGCACAG | 6. |
| HPV15 E6 | forward: TGCAGTTAATTTGGACTGAGGA | 7. |
| | reverse: ATTCAAACTGCGCTGTAGCA | 8. |
| KRT14 | forward: TTTGGCGGCTGGAGGAGGTCACA | 9. |
| | reverse: ATCGCCACCTACCGCCGCCTG | 10. |
| S100A8 | Probe: 56-FAM/ | 11. |
| | ACTTGCCCC/ZEN/ACCAGGTCTTCTG/31ABkFQ | |
| | Primer 1: GCATGTCTCTTGTCAGCTGT | 12. |
| | Primer 2: CGTCGATGATAGAGTTCAAGGC | 13. |
| S100A9 | Probe: 56-FAM/AGCTCTTTG/ZEN/ | 14. |
| | AATTCCCCCTGGTTCA/31ABkFQ | |
| | Primer 1: CAACACCTTCCACCAATACTCT | 15. |
| | Primer 2: CCTCCATGATGTGTTCTATGACC | 16. |
| S100A14 | Probe: 56-FAM/CTGTGTCTG/ZEN/GTCCTTTG | 17. |
| | GTGAGAGT/31ABkFQ | |
| | Primer 1: ACAGATCATGAGCCATCAGC | 18. |
| | Primer 2: ACTGAATTCCTGAGCATCCTC | 19. |
| S100A16 | Probe: 56-FAM/AGCTGGAGA/ZEN/AGGCAGTCAT | 20. |
| | TGTCC/31ABkFQ | |
| | Primer 1: CTGGAGAGGAGGCAGACT | 21. |
| | Primer 2: CTTGTTCTTGACCAGGCTGTA | 22. |
| MMP13 | Probe: 56-FAM/TGAAGACCC/ZEN/CAACCCTA | 23. |
| | AACATCCAAA/31ABkFQ | |
| | Primer 1: AGCCACTTTATGCTTCCTGA | 24. |
| | Primer 2: TGGCATCAAGGGATAAGGAAG | 25. |
| IL24 | Probe: 56-FAM/TCATGTCTT/ZEN/GTCCCT | 26. |
| | CTGGTCCTGT/31ABkFQ | |
| | Primer 1: CCTCTGATTGGTGAATGGTGA | 27. |
| | Primer 2: GGTGTTAAATTGGCGAAAGCA | 28. |
| IL1B | Probe: 56-FAM/AGAAGTACC/ZEN/TGAG | 29. |
| | CTCGCCAGTGA/31ABkFQ | |
| | Primer 1: CAGCCAATCTTCATTGCTCAAG | 30. |
| | Primer 2: GAACAAGTCATCCTCATTGCC | 31. |
| HSPD1 | Probe: 56-FAM/CCTTCCTTG/ZEN/GCTATAG | 32. |
| | AGCGTGCC/31ABkFQ | |
| | Primer 1: GCACTACCACTGCTACTGT | 33. |
| | Primer 2: AGTTCAGCAATTACAGCATCAAC | 34. |

Wart Development

For 10 weeks following viral infection or sham infection, mice were monitored for the development of warts. As previously described,³⁷ mice with warts lasting >2 months were considered to have “persistent” warts. We classified these mice as “nonimmune” and they were excluded from chemical and UV carcinogenesis studies. Mice that showed either no wart development or wart rejection were classified as “immune” and were entered into carcinogenesis studies.

T Cell Isolation and Transfer

MumPV1-colonized FVB mice that never developed warts or exhibited spontaneous regression of warts by 10 weeks following infection (immune mice) were used as T cell donors. A single cell suspension of CD4⁺ and CD8⁺ T cells from skin-draining lymph nodes were prepared using EasySep™ Mouse T Cell Isolation Kit (Stemcell Technologies, Vancouver, Canada, catalog no. 19851). 1 million T cells in 200 μl sterile normal saline were injected intravenously into the tail vein of wart-bearing (nonimmune) Wt FVB mice. The recipient mice were monitored for the resolution of their skin warts. To assess the MmuPV1-specific nature of T cells from MumPV1-colonized immune mice, we transferred their sorted CD4⁺ and CD8⁺ T cells from skin-draining lymph nodes into CD4^{-/-}; CD8^{-/-} mice as recipient mice. Donor mice were injected intravenously with 2 g CD45-APC (BioLegend, San Diego, Calif., catalog no. 103112) 3 minutes prior to harvest to exclude any circulating naïve T cells. At harvest, single cell suspensions of skin-draining lymph nodes were stained with CD3e-PE-Cy7 (Biolegend, catalog no. 100320), CD4-APC-Cy7 (Biolegend, catalog no. 100414), CD8α-FITC (Biolegend, catalog no. 100706, Table 5), and CD62L-PerCP/Cy5.5 (Biolegend, catalog no. 104432, Table 5). Sorted CD45⁻ CD3⁺ CD4⁺ CD62L^{low} and CD45⁻ CD3⁺ CD8⁺ CD62L^{low} donor memory T cells³⁸ were injected intravenously into CD4^{-/-}; CD8^{-/-} mice at 129,600 cells per mouse (6:1 CD4⁺:CD8⁺ ratio) in 200 μl sterile normal saline. As a control for MumPV1-specific T cells, a group of Wt FVB mice were vaccinated against an unrelated virus (mouse parvovirus type 1) in order to propagate a population of T cells that would not respond to MmuPV1. This group of T cell donors was vaccinated with a cocktail of 50 ug polyinosinic-polycytidylic acid (poly(I:C), Sigma Aldrich, St. Louis, Mo., catalog no. P1530) combined with mouse parvovirus virus-like particles (VLPs) in 200 μl of sterile normal saline delivered via subcutaneous injection at four sites (50 μl per site per vaccination) on the back skin at 30 days and 3 days prior to harvest. 200 μl of 5% Imiquimod (Sigma-Aldrich, catalog no. 1338313) dissolved in dimethyl sulfoxide (DMSO) and diluted in 100% EtOH (Sigma-Alrich, catalog no. 276855) was applied topically following each vaccination. T cell recipients and T cell-deficient CD4^{-/-}; CD8^{-/-} mice and Wt FVB mice were infected with MmuPV1 two days following T cell transfer, including mice that received T cells from parvovirus vaccine plus a topical imiquimod-treated donors. Another subgroup of MmuPV1-T cell recipients, T cell-deficient CD4^{-/-}; CD8^{-/-} and Wt mice received a SCC cell line injection into their right flank and monitored for tumor growth (FIG. 7A). Mice were monitored closely for wart development in MmuPV1 infection cohorts and SCC growth in tumor cohorts for two months including pictures and tumor size measurements. To examine the presence/absence of T cells in the recipient mice, peripheral blood was collected from the mice 3 weeks following the T cell transfer. 2-3 drops of blood per mouse via submandibular vein was collected in 10 ml of RBC Lysis Buffer (Biolegend, catalog no. 420301), stained with CD3e-PE-Cy7, CD4-APC-Cy7 and CD8α-FITC, and examined by flow cytometry.

TABLE 5
Antibodies and primers used for tissue analysis.

| Immunostaining | Clone | Company |
|---|---|---|
| Rat Anti-Mouse CD3 | CD3-12 | Abcam, Cambridge, MA |
| Rabbit Anti-Mouse CD4 | EPR19514 | Abcam |
| Rabbit Anti-Mouse CD8 | D4B9C | Cell Signaling Technology, |
| | | Danvers, MA |
| Secondary Antibodies | | |
| and staining kits | | |
| Goat anti-Rabbit IgG, | | Thermo Fisher Scientific, |
| Alexa Fluor ® 488 | | Waltham, MA |
| Goat anti-Mouse IgG, | | Thermo Fisher Scientific |
| Alexa Fluor ® 568 | | |
| Flow Cytometry | | |
| Mouse | | |
| CD3e-PE-Cy7 | 145-2C11 | BioLegend, San Diego, CA |
| CD4-APC-Cy7 | RM4-5 | BioLegend |
| CD8α-FITC | 53-6.7 | BioLegend |
| CD45-APC | 30-F11 | BioLegend |
| CD62L- PerCP/Cy5.5 | MEL-14 | BioLegend |
| Human | | |
| CD3-FITC | UCHT1 | BD Biosciences, San Jose, |
| | | CA |
| CD4-APC eflour780 | RPA-T4 | eBioscience |
| CD8-PerCP Cy5.5 | SK1 | BioLegend |
| CD45-APC | HI30 | BioLegend |
| CD69-BV421 | FN50 | BioLegend |
| CD107a-BV605 | H4A3 | BioLegend |
| CD137-BUV395 | 4B4-1 | BD Biosciences |

Chemical Carcinogenesis Protocol

Following infection and evidence of MmuPV1 immunity, C57BL/6J and FVB mice underwent a skin chemical carcinogenesis protocol. All animals were shaved and 7 days later received a single dose of 100 g 7,12-dimethylbenz(a)anthracene (DMBA) (Sigma Aldrich, catalog no. D3254) in 200 μl acetone on the back skin. One week later, treatments with 12-O-tetradecanoylphorbol-13-acetate (TPA) (Sigma Aldrich, catalog no. P1585) dissolved in 200 μl acetone were initiated (3× per week for 30 weeks in C57BL/6J and 2× per week for 20 weeks in FVB cohorts). Throughout the carcinogenesis protocol, tumors were counted every week and pictures were collected every other week. Final tumor burden was determined based on the total number of palpable skin lesions developed on the animals' back skin.

UV Carcinogenesis Protocol

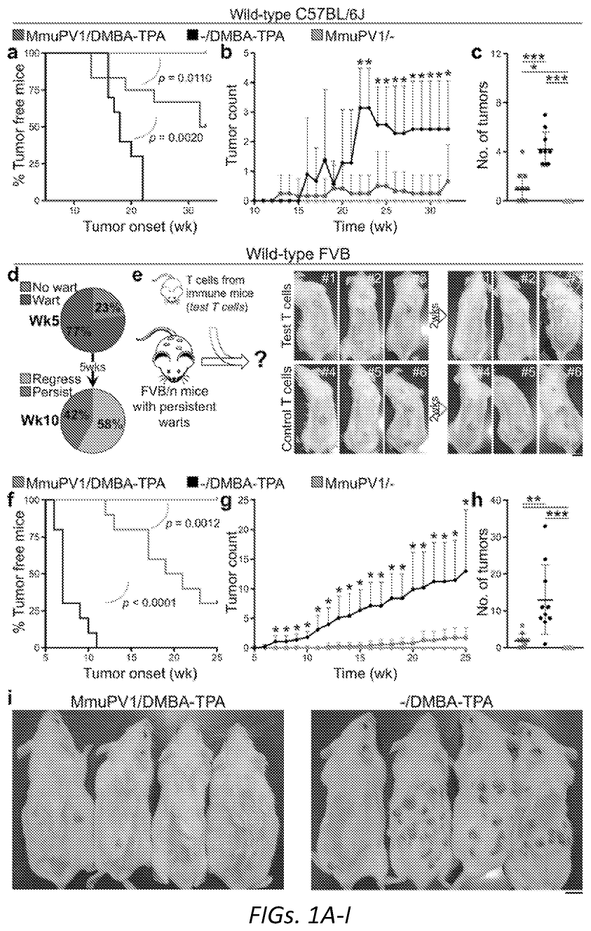

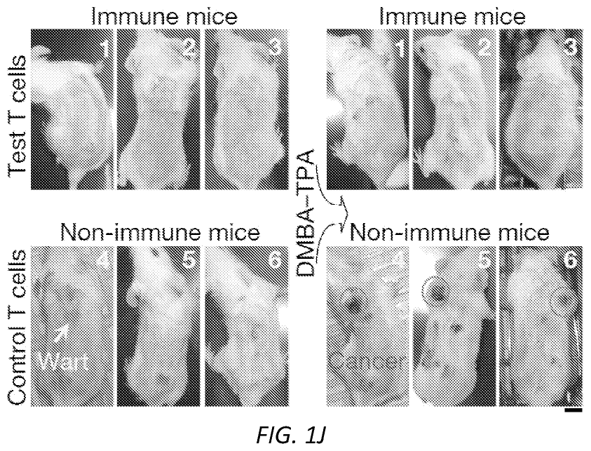

Following infection and evidence of MmuPV1 immunity, SKH-1 mice underwent a skin carcinogenesis protocol (FIG. 1 IG). Mice received a single dose of 50 ug DMBA in 200 μl acetone on the back skin. One week later, SKH-1 mice received 25 weeks of narrow-band ultraviolet B (UVB) (302-312 nm) three times weekly via UVP Black-Ray® Lamp UVB (VWR, Radnor, Pa., catalog no. 36575-052), which was periodically calibrated using International Light IL 1400A Digital Lightmeter. Mice received 100 mJ/cm² UVB at each UV treatment timepoint. This is considered a suberythemic dose for a fair-skinned individual of average tanning ability (Fitzpatrick skin types 2-3), which approximates 40-60 min of sun exposure in Florida midday in the summer.^{39,40} Throughout this carcinogenesis protocol, tumors were counted every week and pictures were collected every other week. Final tumor burden was determined based on the total number of palpable skin lesions developed on the animals' back skin.

Histology and Immunofluorescence Staining

Dorsal skin samples were harvested and fixed in 4% paraformaldehyde (PFA, Sigma Aldrich, catalog no. P6148) overnight at 4° C. Next, tissues were dehydrated in ethanol, processed, and paraffin embedded. Five m sections of paraffin-embedded tissue were cut, deparaffinized, and stained with hematoxylin and eosin (H&E). For immunofluorescence staining, rehydrated tissue sections were permeated with 1×PBS supplemented with 0.2% v/v Triton X-100 (Thermo Fisher Scientific, Waltham, Mass., catalog no. BP151) for 5 min. Antigen retrieval was performed in Antigen Unmasking Solution (Vector Laboratories, Burlingame, Calif., catalog no. H-3300) using a Cuisinart pressure cooker for 20 min at high pressure. Slides were washed 3× for 3 min in 1×PBS supplemented with 0.1% v/v Tween 20 (Sigma-Aldrich, catalog no. P1379). Sections were blocked with 5% m/v bovine serum albumin (Fisher Scientific, Hampton, N.H., catalog no. BP1600) and 5% v/v goat serum (Sigma-Aldrich, catalog no. G9023). The slides were stained overnight at 4° C. with 1:500 rat anti-CD3 and either 1:500 rabbit anti-CD4 or 1:400 rabbit anti-CD8a (Table 5). The following day, slides were washed as above and incubated for 2 hr at room temperature with 1:200 goat anti-rat-PE and 1:500 goat anti-rabbit-FITC (Table 5). After washing as above, slides were incubated with 1:4000 DAPI (Invitrogen, Carlsbad, Calif., catalog no. D3571) for 5 min at room temperature, then washed as above. Slides were mounted with Prolong Gold Antifade Reagent (Invitrogen, catalog no. P36930). Once stained, ten randomly selected images of morphologically normal skin at 200× total magnification were obtained of each section. Blinded manual counting of CD3⁺, CD4⁺ and CD8⁺ cells were performed using the ZEN Blue ‘event’ tool (Zeiss, Oberkochen, Germany). Positive cells were determined by comparing fluorescent intensity to the background, which was minimized using ZEN. Analysis was performed based on the number of double positive cells (e.g. CD3⁺ CD4⁺) in the epithelial compartments (i.e., epidermis and hair follicle) or dermis and the total number of CD3⁺ cells in each image.

Serology

Using methods described previously,⁴¹ anti-MmuPV1-specific antibodies in mouse serum were detected via enzyme-linked immunosorbent assay (ELISA).

RNA and DNA In Situ Hybridization

RNAish and DNAish were performed on formalin fixed paraffin embedded (FFPE) human and mice tissue sections using RNAscope® probes and protocols (Supplementary Table 2; DNA probes were generated using the sense strand of viral DNA at the same RNA probes binding sites; Advanced Cell Diagnostics, California, USA).⁴² We used the HybEZ™ Hybridization System to perform RNAscope® Assay hybridization and incubation steps. Briefly, sections with 5 m thickness were baked in a dry oven for 1 hr at 60° C. and immediately deparaffinized in xylene, followed by rehydration in an ethanol series. Epitope retrieval was performed by placing the slides in RNAscope® 1× Target Retrieval Reagent (Advanced Cell Diagnostics, catalog no. 322000) in 102° C. for 15 mins and then washed. Protease treatment was performed next by adding RNAscope® Protease Plus (Advanced Cell Diagnostics, catalog no. 322331) to the section and incubated at 40° C. for 30 min in a HybEZ™ Oven II (Advanced Cell Diagnostics, catalog no. 321720). After probe hybridization with target probes, preamplifier and amplifier, sections were stained with Fast RED reagent (RNAscope® 2.5 HD Detection Reagents—RED, Advanced Cell Diagnostics, catalog no. 322360). 50% Hematoxylin plus 0.02% Ammonia water was used as Counterstain. Positive and negative probes were used in each assay to ensure proper controls. We used probes to an endogenous housekeeping gene peptidylprolyl isomerase B (PPIB, Advanced Cell Diagnostics, catalog no. 313901) and the bacterial gene dapB (Advanced Cell Diagnostics, catalog No. 310043) as positive and negative controls, respectively. We assessed RNAish and DNAish red signals under a standard bright field microscope at 400× magnification. Ten representative areas of skin cancer and normal skin from each slide were imaged at 400× magnification and positive RNAish/DNAish signals and keratinocyte nuclei were counted in each image in a blinded manner.

qRT-PCR

RNA samples were extracted from human tissues that were stored in Allprotect (Qiagen, catalog no. 76405) at 4° C. and flash frozen samples stored at −80° C. A piece of tissue (˜50-100 mg) was washed using sterile 1×PBS and placed into tube containing a 5 mm TissueLyser bead. Following this 600 μl of RLT and PME was added to the sample and bead. The tissue was homogenized for 5 minutes through mechanical manipulation. The liquid was transferred into a new tube where 1 ml of TRIzol was added. Using standard Thermo Fisher protocols for TRIzol, the solution was mixed and centrifuged at 4° C. for 10 minutes. The clear supernatant was collected and 0.2 ml of chloroform/1 mL of TRIzol was added. The mixture is centrifuged and the clear supernatant is retrieved. For extraction of RNA we used the Allprep DNA/RNA mini kit (Qiagen, catalog no. 80284). The clear supernatant is then added to the Allprep DNA spin column, the flow through was mixed with 1 volume of 70% ethanol. This solution is mixed and applied to the RNAeasy spin column where standard methods of purification are followed including DNase digestion. RNA was quantified using nanodrop and 1 pg of RNA was used for reverse-transcriptase reaction using SuperScript III RT Kit (ThermoFisher, catalog no. 18080044). 1 μg of RNA was mixed with 0.25 mg/ml random primers, 10 mM dNTP mix and nuclease free H2O for a total of 13 μl. This sample was incubated at 65° C. for 5 minutes. A mix of diluted 1× first strand buffer, 0.1M of DTT, 40U/μl of RNaseOUT and superscript III 200U was added to the nucleotide mix. The sample was then incubated in a thermocycler. The program consisted of 5 min at 25° C., 1 hour at 50° C., and 15 min at 70° C. Following PCR, cDNA samples were diluted 1:9 using UltraPure™ DNase/RNase-Free Distilled. 3 μl of the 1:9 dilution was used in the total 10 μl qPCR reaction. For forward and reverse primer 0.5p of 10 μM concentration was used. Primers were purchased through IDT.⁴³ 5 μl of SYBR® Green master mix was used along with 1 μl of UltraPure™ DNase/RNase-Free Distilled Water per reaction. The qPCR was run on LightCycler 480 II (Roche, Basel, Switzerland, product no. 05015278001). qRT-PCR products were verified by running them on a 1% agarose gel at 120V for 60 minutes.

Human T Cell Isolation and Peptide Stimulation

T cells were isolated from human skin as previously described.⁴⁴ Briefly, discarded normal facial skin samples generated as part of Mohs surgery repair was obtained. subcutaneous fat tissue was removed from human facial skin tissue, and remaining tissue was minced. Small fragments of tissue were digested in RPMI 1640 including 1% DNase-I (Sigma-Aldrich) and 0.2% collagenase-I (Fisher Scientific) for 2 hr at 37° C. Then cells were collected through 40 μm cell strainer, and were incubated in RPMI 1640 including 20% FBS, 1% penicillin/streptomycin, 1% glutamine, 0.00035% 2-mercaptoethanol, and 50U/ml human IL-2 recombinant (BioLegend). Human skin T cells were seeded in 96 well plate and treated with a pool of 5 β-HPV E7 peptides (HPV5/8/9/20/38, 5 μg/mL of each peptide, custom peptides, JPT, Berlin, Germany), pool of HPV16 E7 peptides (5 μg/mL of each peptide, PepMix™ HPV 16 (Protein E7), JPT, product code PM-HPV16-E7) or 50 ng/ml phorbol 12-myristate 13-acetate (PMA) plus 500 ng/ml Ionomycin (Ion). Peptide pools were generated as 15mers with 11 amino acid overlap across the length of E7 proteins. After 24 hr of peptide exposure, cells were collected and stained with antibodies to surface markers for T cell activation (Table 5), and examined by flow cytometry (BD LSRFortessa X-20). Flow data were analyzed using FlowJo software Ashland, Oreg.).

### Example 1. Immunity to Commensal Papillomaviruses Protects Against Skin Cancer

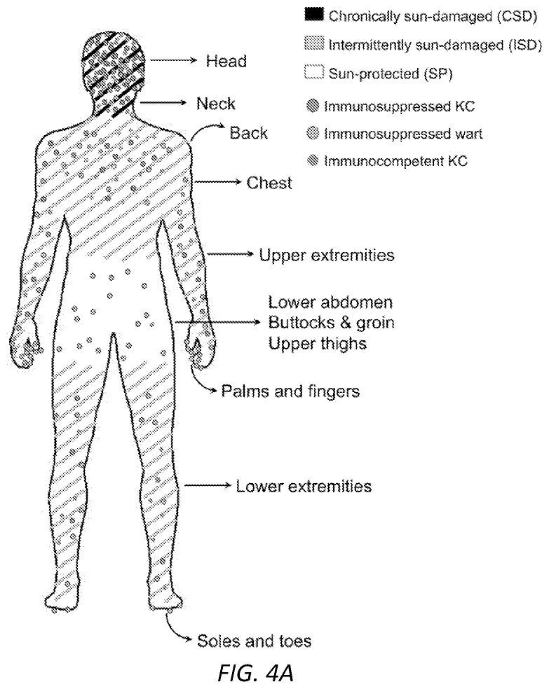

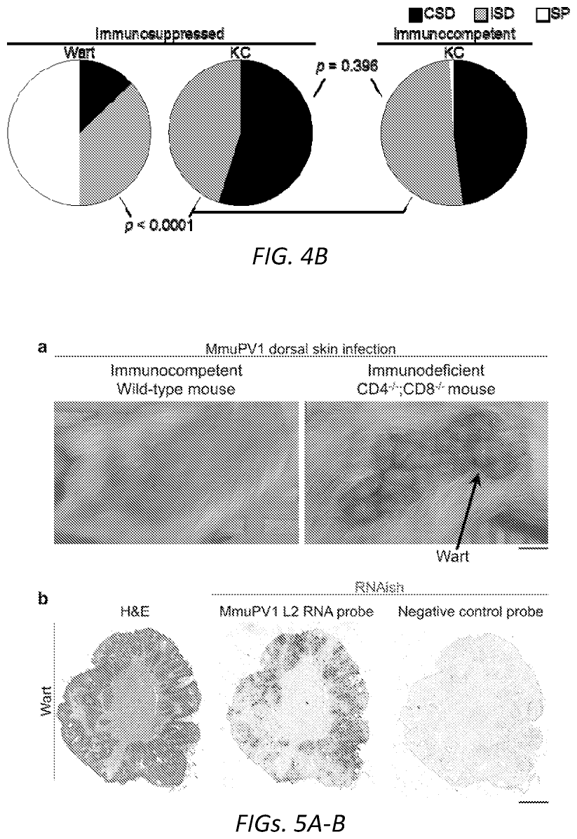

To investigate whether cutaneotropic HPVs contribute to skin cancer development, we performed a clinical study in which the anatomical localization of skin cancers and warts from 83 immunosuppressed participants were mapped. Although 86% of HPV-driven warts developed on the sun-protected (SP) and intermittently sun-damaged (ISD) skin, 41 out of 74 (55%) skin cancers developed on chronically sun-damaged skin of head and neck (Tables 1A-B and FIGS. 4A-B). In contrast, the anatomical distribution of skin cancers in immunosuppressed patients matched those of immunocompetent patients (Tables 1A-B and FIGS. 4A-B). The absence of an increase in skin cancer propensity on the anatomical sites with high viral activity and wart development suggested that UV was the dominant skin cancer initiator in immunosuppressed patients.

TABLE 1A
Demographics and skin lesions distribution. Note that
the immunosuppressed and immunocompetent patients in
the study are matched based on their skin cancer types.

| | Immuno- | | Immuno- | |
|---|---|---|---|---|
| | suppressed | | competent | P |
| | (n = 83) | | (n = 66) | value |
| Age, mean (SD), y | 60 | (13) | 68 (11) | 0.0002 |
| (Range) | (21-87) | | (37-91) | |
| Gender, n (%) | | | | >0.9999 |
| Male | 55 | (66) | 43 (65) | |
| Female | 28 | (34) | 23 (35) | |
| Reason for Immunosuppression, n (%) | | | | |
| OTR, Kidney | 38 | (46) | — | |
| OTR, Lung | 21 | (25) | — | |
| OTR, Liver | 7 | (8) | — | |
| OTR, Heart | 4 | (5) | — | |
| Leukemia | 5 | (6) | — | |
| Other* | 8 | (10) | — | |
| Skin cancers, n | | | | 0.6913 |

| | | |
|---|---|---|
| BCC | 15 | 17 |
| SCC | 59 | 55 |
| Warts**, n | 83 | — |

*patients who are on systemic immunosuppressive medications due to inflammatory diseases like ulcerative colitis.
**wart cluster on a single anatomical site is counted as one.
CSD: chronically sun-damaged skin, ISD: intermittently sun-damaged skin, SP: sun-protected skin.

TABLE 1B
Anatomic distribution of skin cancers and warts in the patient population
from Table 1B. This table defines the anatomic regions referred
to as chronically sun-damaged skin (CSD), intermittently sun-damaged
skin (ISD) and sun-protected skin (SP) in Table 1. Incidence of
warts and skin cancers in each region are provided.

| | |
|---|---|
| Immuno- | Immuno- |
| suppressed | competent |
| (n = 83) | (n = 66) |

| Skin cancer, n (%) | | | | |
|---|---|---|---|---|
| Chronically sun-damaged skin | | | | |
| Head | 37 | (50) | 30 | (42) |
| Neck | 4 | (5) | 4 | (6) |
| Intermittently sun-damaged skin | | | | |
| Upper extremities | 10 | (14) | 9 | (12.5) |
| Chest | 12 | (16) | 9 | (12.5) |
| Back | 5 | (7) | 9 | (12.5) |
| Lower extremities | 6 | (8) | 10 | (14) |
| Sun-protected skin | | | | |
| Lower Abdomen | 0 | (0) | 0 | (0) |
| Upper thighs | 0 | (0) | 0 | (0) |
| Buttocks and groin | 0 | (0) | 1 | (1) |
| Palms and fingers | 0 | (0) | 0 | (0) |
| Soles and toes | 0 | (0) | 0 | (0) |
| Wart, n (%) | | | | |
| Chronically sun-damaged skin | | | | |
| Head | 8 | (10) | — | |
| Neck | 3 | (4) | — | |
| Intermittently sun-damaged skin | | | | |
| Upper extremities | 16 | (19) | — | |
| Chest | 5 | (6) | — | |
| Back | 1 | (1) | — | |
| Lower extremities | 9 | (11) | — | |
| Sun-protected skin | | | | |

| | | | |
|---|---|---|---|
| Lower Abdomen | 5 | (6) | — |
| Upper thighs | 7 | (8) | — |
| Buttocks and groin | 5 | (6) | — |
| Palms and fingers | 19 | (23) | — |
| Soles and toes | 5 | (6) | — |

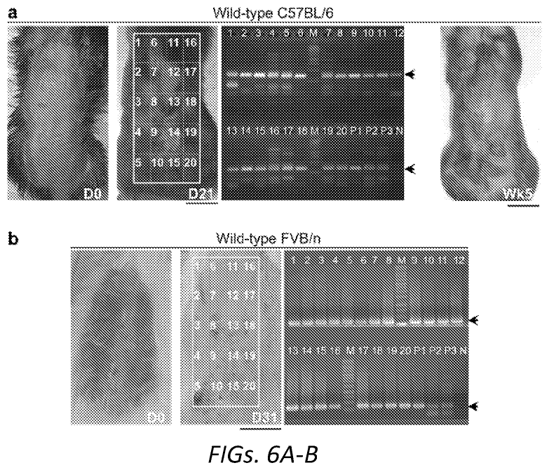

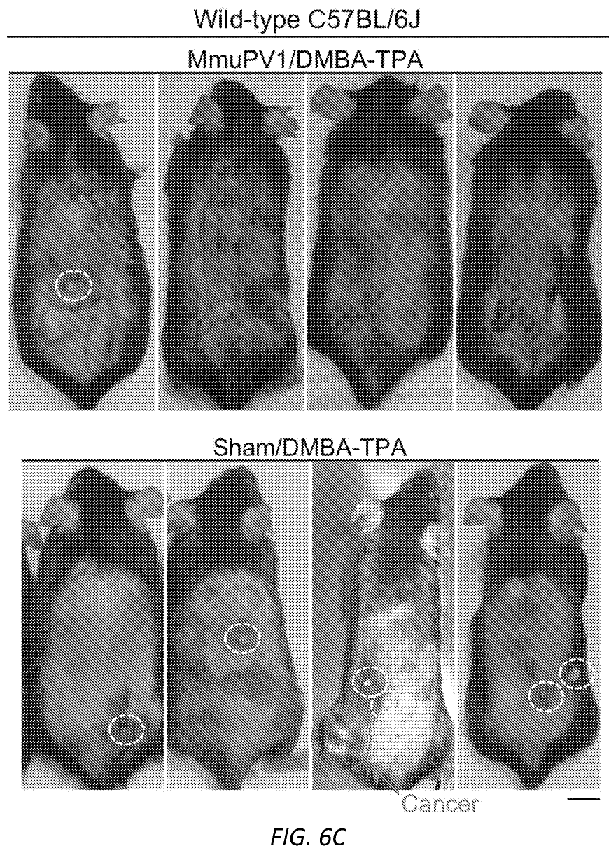

In order to determine the impact of papillomavirus on carcinogen-driven skin cancer, we utilized the mouse papillomavirus (MmuPV1), which has recently emerged as a robust tool in the study of HPV-related skin disease.^{6,7} We developed a method to infect the back skin of animals with MmuPV1, which led to a confluent wart development on the back skin of T cell-deficient, CD4^{-/-}; CD8^{-/-} mice, but no skin lesions in immunocompetent, Wt animals (FIGS. 5A-B). The infection of Wt C57BL/6 mice with MmuPV1 led to a broad colonization of their back skin with no wart development in 100% of the animals (FIG. 6A). Two months after infection, MmuPV1- and sham-infected mice were subjected to a standard skin chemical carcinogenesis protocol on the back skin using dimethylbenz(a)anthracene (DMBA) once and 12-O-tetradecanoylphorbol-13-acetate (TPA) treatment three times a week for 30 weeks.²⁰ Surprisingly, animals that were colonized with MmuPV1 showed a significant delay in the onset of carcinogen-induced skin tumors compared to uninfected mice (p=0.0020; FIG. 1A). In addition, MmuPV1-colonized mice developed significantly fewer tumors over time (p<0.05 started at week 22 post DMBA; FIG. 1B) and completed the study with significantly less tumor burden compared to uninfected mice (p<0.0001; FIG. 1C, 6C). MmuPV1-infected Wt C57BL/6J mice that did not receive DMBA-TPA remained skin tumor/wart free for the 32-week follow-up period (FIG. 1A-C).

To investigate the impact of papillomavirus on skin cancer in the FVB strain of mice, which are more prone to chemical skin carcinogenesis,²⁰ we infected the back skin of Wt FVB mice as described above and achieved complete skin colonization (FIG. 6B). 23% of the infected mice showed complete immunity immediately after infection. However, 77% of the mice developed warts on their back skin at 5 weeks post-infection (FIG. 1D). At 10 weeks post-infection, warts completely regressed in 58% of wart-bearing FVB mice, indicative of an antiviral adaptive immunity. 42% of the animals continued to have persistent warts with no change in their wart numbers (FIG. 1D). T cells transferred from the skin-draining lymph nodes of MmuPV1-immune mice (i.e., no warts) rendered immunity to mice with persistent warts, leading to wart rejection in 2 weeks following the adoptive T cell transfer (FIG. 1E). To examine whether memory T cells isolated from the immune mice were MmuPV1-specific, we transferred these T cells to wart-prone CD4^{-/-}; CD8^{-/-} mice followed by MmuPV1 infection of their back skin (FIG. 7A). CD4^{-/-}; CD8^{-/-} mice that received T cells from MmuPV1-immune mice developed fewer warts following infection compared to T cell-deficient CD4^{-/-}; CD8^{-/-} mice and CD4^{-/-}; CD8^{-/-} mice that received T cells from Wt mice treated with a parvovirus vaccine (FIG. 7B). In contrast to their protective effect against MmuPV1-driven warts, T cells from MmuPV1-immune mice did not impact a SCC tumor growth in CD4^{-/-}; CD8^{-/-} mice (FIG. 7C).

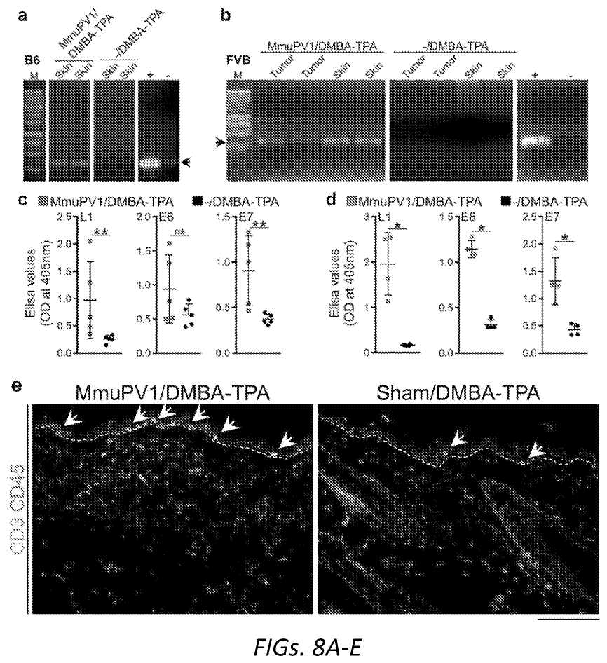

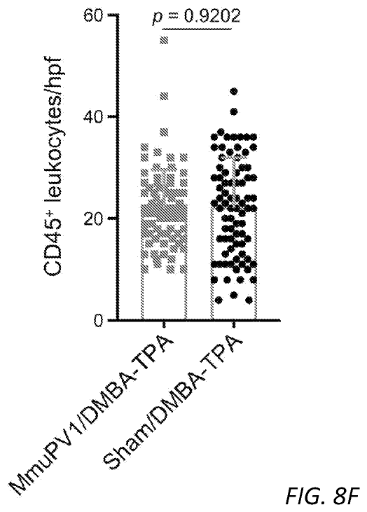

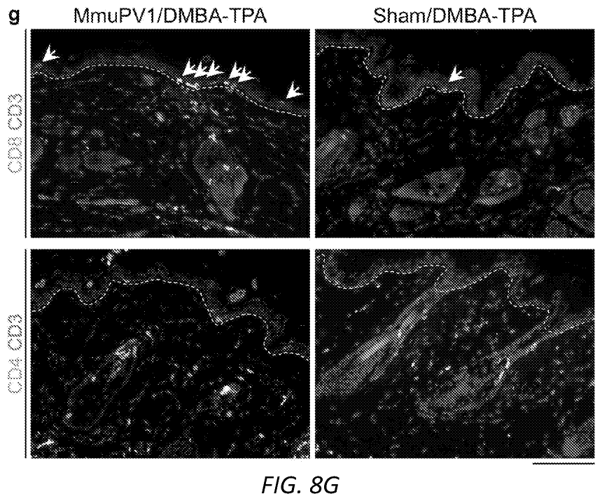

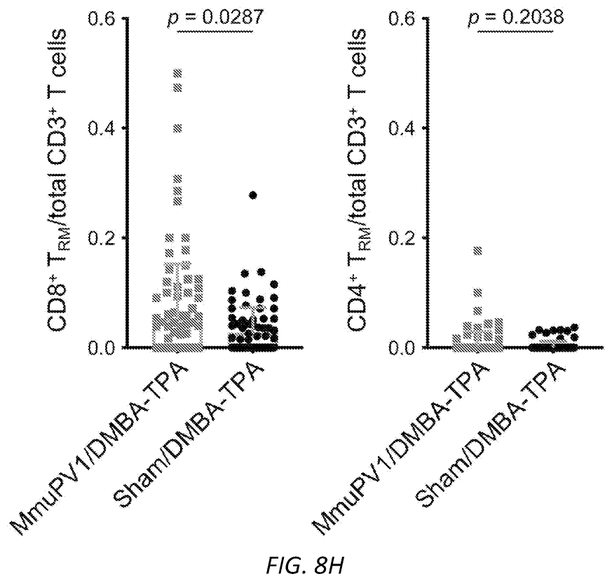

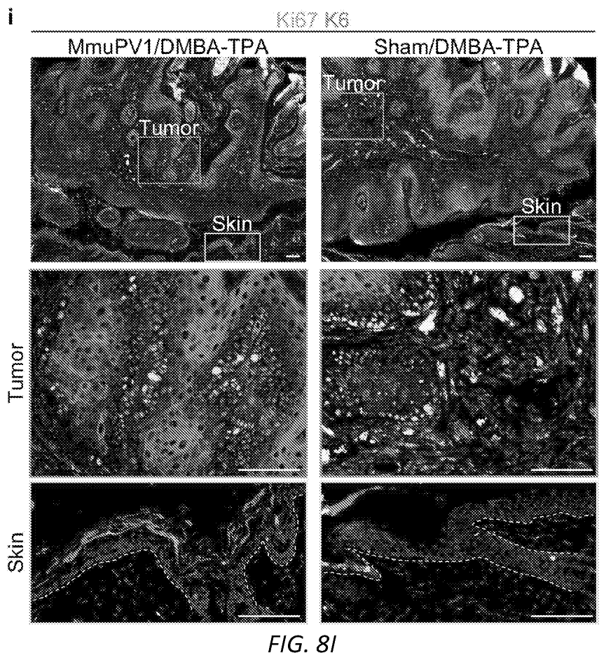

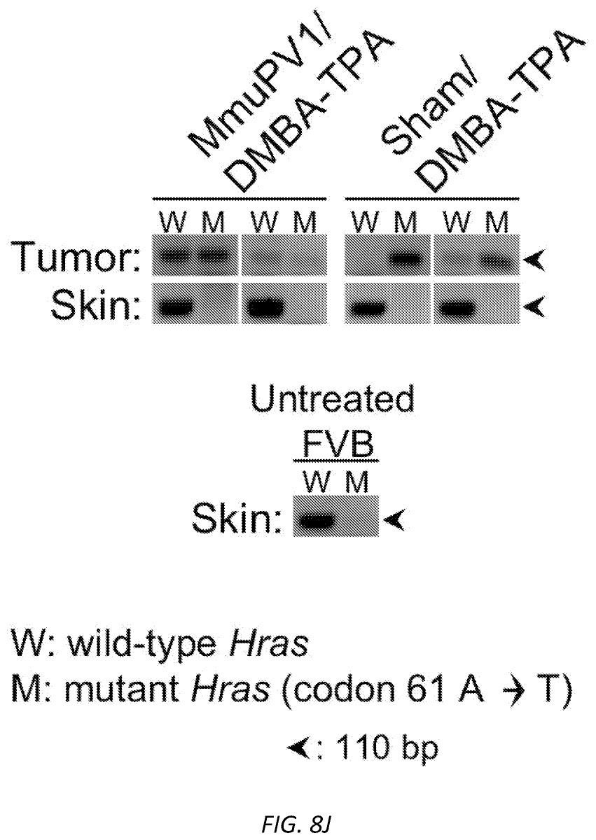

MmuPV1-colonized Wt FVB mice with natural or acquired immunity against MmuPV1 were treated with DMBA once followed a week later with twice weekly treatment with TPA for 20 weeks. Similar to C57BL/6J animals, Wt FVB mice colonized with MmuPV1 were protected against chemical carcinogenesis and showed a significant delay in skin tumor onset (p<0.0001; FIG. 1f). MmuPV1-colonized mice developed fewer tumors over time (p<0.05 started at week 7 post DMBA; FIG. Ig) and they had markedly less tumor burden at the completion of the study p<0.01; FIGS. 1h and i). Thus, MmuPV1-colonized immune FVB mice that received 7,12-dimethylbenz[a]anthracene (DMBA) and 12-O-tetradecanoylphorbol-13-acetate (TPA) for 20 weeks were protected against chemical carcinogenesis compared with sham-infected mice. Furthermore, mice with acquired immunity after T cell transfer were also protected from chemical carcinogenesis (FIG. 1J). We detected MmuPV1 viral DNA in the normal skin (FIGS. 8A and B) and anti-MmuPV1 L1, E6 and E7 antibodies in the blood (FIGS. 8C and D) of MmuPV1-colonized Wt C57BL/6J and FVB mice at the completion of the DMBA-TPA protocol. Importantly, MmuPV1-colonized mice showed increased homing of CD8⁺ T cells into the epithelial compartment of the skin compared to uninfected mice as shown by CD8⁺ tissue resident memory T (TRM) to total CD3⁺ T cell ratios (p=0.0004, FIGS. 5E-H). DMBA-TPA-induced skin tumors in MmuPV1-colonized mice showed similar proliferative and mutational signatures to those in sham-infected mice and lacked MmuPV1 viral transcripts (FIGS. 8I-J, 9). We did not detect any changes in CD4⁺ T cell populations in the MmuPV1-infected versus uninfected skin (FIGS. 8E and F). Interestingly, we were able to detect MmuPV1 RNA in normal skin of MmuPV1-colonized mice at the time of harvest (FIG. 9). These findings demonstrate the protective effect of anti-MmuPV1 T cell immunity in suppressing carcinogen-driven tumors in MmuPV1-colonized skin.

To determine the effect of papillomavirus colonization on UV carcinogenesis, we studied hairless SKH-1 mice that are immunocompetent and develop skin tumors in response to UV radiation.²¹ Following MmuPV1 infection, abundant MmuPV1 L2 RNA was detectable in the skin of the immune mice (i.e., no wart development) and in the skin and warts of the animals lacking immediate anti-MmuPV1 immunity at 3 weeks after the infection (FIG. 7A). MmuPV1-infected immune mice8 that received a single immunosuppressive dose of ultraviolet light B (UVB; 300 mJ cm-2) at three months after MmuPV1 infection developed warts9, indicating the long-term persistence of MmuPV1 colonization of the skin (FIGS. 10B-C). To avoid immunosuppressive UV exposure, MmuPV1- and sham-infected mice were treated with DMBA a week before undergoing treatment with UVB (100 mJ cm-2) three times a week for 25 weeks. A small subset of SKH-1 mice that had persistent warts two months after MmuPV1 back-skin infection (Wang et al., PLoS Pathog. 11, e1005243 (2015)) were vaccinated with MmuPV1 live virus particles intraperitoneally three times over a two-week period. Four weeks later, five out of nine mice developed immunity against MmuPV1, as demonstrated by the rejection of their persistent warts (FIG. 10D). The mice with acquired immunity against MmuPV1 developed markedly fewer skin tumors compared with the non-immune mice (P=0.0159; FIGS. 10D-E). Furthermore, the total numbers of T cells and CD8+ T cells were markedly increased in skin tumors of MmuPV1-colonized mice (FIGS. 10F-M, 11A-C). The levels of skin and tumor-infiltrating CD3-CD45+ leukocytes and CD4+ T cells were unchanged between the two groups (FIGS. 10F-M, 11D-F).

To determine the role of CD8+ T cells in mediating the anti-tumor immunity induced by papillomavirus skin colonization, SKH-1 mice were infected with MmuPV1 or sham-infected with MmuPV1 virus-like particles (sham(VLP)). MmuPV1- and sham(VLP)-infected mice underwent CD8+ T cell depletion, mediated by anti-CD8 antibodies, together with the UV carcinogenesis protocol (FIGS. 11 G-H). Notably, MmuPV1-colonized SKH-1 mice that were treated with IgG control developed markedly fewer tumors compared to the MmuPV1-colonized mice that underwent T cell depletion, and compared with both the IgG- and anti-CD8-antibody-treated control groups that were infected with sham(VLP) (FIGS. 11I-J). Consistent with our findings in other immunocompetent strains of mice, MmuPV1-colonized Xpc−/−mice which are deficient in the ability to repair UV-induced DNA mutations (Sands et al., Nature 377, 162-165 (1995))—were protected from skin cancer compared to their sham-infected controls (FIGS. 11K-N).

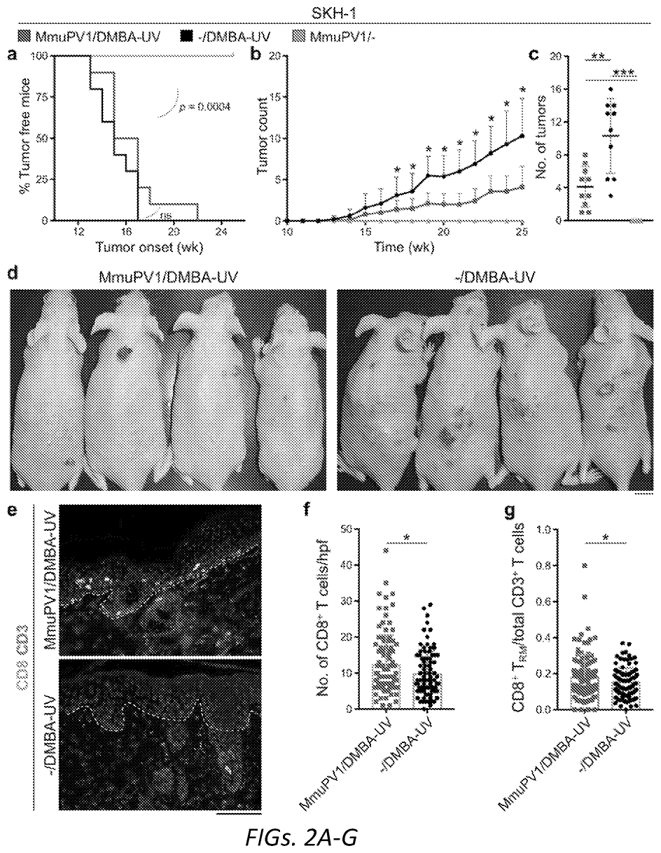

To avoid high immunosuppressive UV dosing,²³ the back skin was treated with one dose of 50 g DMBA a week prior to receiving three times per week 100 mJ/cm² UVB treatment for 25 weeks. Although the skin tumor onset was not significantly delayed (FIG. 2A), MmuPV1-colonized SKH-1 mice developed significantly fewer tumors over time (p<0.05 started at week 17 post DMBA; FIG. 2B) and had markedly less tumor burden at the completion of the study compared to the uninfected mice (p<0.005; FIGS. 2C and D). Interestingly, the widespread epidermal dysplasia caused by DMBA-UV treatment in the back skin of the uninfected mice was absent in MmuPV1-colonized animals (FIG. 12A). This block in DMBA-UV-induced dysplasia was reflected by reduced epidermal thickness in MmuPV1-colonized mice compared to their uninfected counterparts (FIG. 12B). The immune cell analysis revealed a significant increase in total number of CD8⁺ T cells and CD8⁺ T_{RM}/total T cell ratio in the skin of MmuPV1-colonized mice compared to their uninfected controls at the completion of DMBA-UV protocol (p<0.05, FIG. 2e-g). No significant difference in the total number of CD4⁺ T cells was observed between groups (FIGS. 12C-D). MmuPV1 DNA was detectable in the normal skin of MmuPV1-colonized mice at the completion of DMBA-UV protocol (FIG. 12E).

The protective effect of anti-MmuPV1 immunity against carcinogen-driven skin cancer in mice suggested that β-HPVs in the skin of the immunocompetent individuals may play a similarly protective role. To examine this, we utilized a pool of β-HPV RNAish probes that detect the E6/7 transcripts of 25 β-HPV types on histological sections providing a novel insight into subcellular localization of the virus in the skin (FIG. 13 and Table 2).¹⁶ β-HPV RNAish detected μ-HPV transcripts in keratinocytes of a wart (positive control) and a skin cancer from an immunosuppressed patient (FIGS. 14A-B). Normal skin from an 18-year-old immunocompetent patient showed no detectable μ-HPV RNAish signal (FIG. 14B). HPV5, 9 and 15 E6 qRT-PCR on RNA isolated from the same skin cancer and normal skin validated the β-HPV RNAish results (FIG. 14B).

TABLE 2
β-HPV and MmuPV1 probes used for RNAish. All β-HPV
probes were combined to create a single pan-β-
HPV probe. DNAish probes are the sense strand on the
viral DNA at the same sites where RNA probes bind.

| Probe name | Catalog # |
|---|---|
| RNAscope ® Probe - HPV5 | 421581 |
| RNAscope ® Probe - HPV8 | 421591 |
| RNAscope ® Probe - HPV9 | 421601 |
| RNAscope ® Probe - HPV12 | 421611 |
| RNAscope ® Probe - HPV14 | 421621 |
| RNAscope ® Probe - HPV15 | 421631 |
| RNAscope ® Probe - HPV17 | 421641 |
| RNAscope ® Probe - HPV19 | 421651 |
| RNAscope ® Probe - HPV20 | 421661 |
| RNAscope ® Probe - HPV21 | 421671 |
| RNAscope ® Probe - HPV22 | 421681 |
| RNAscope ® Probe - HPV23 | 421691 |
| RNAscope ® Probe - HPV24 | 421701 |
| RNAscope ® Probe - HPV25 | 421711 |
| RNAscope ® Probe - HPV36 | 421721 |
| RNAscope ® Probe - HPV37 | 421731 |
| RNAscope ® Probe - HPV38 | 421741 |
| RNAscope ® Probe - HPV47 | 421751 |
| RNAscope ® Probe - HPV49 | 421761 |
| RNAscope ® Probe - HPV75 | 421771 |
| RNAscope ® Probe - HPV76 | 421781 |
| RNAscope ® Probe - HPV80 | 421791 |
| RNAscope ® Probe - HPV92 | 421801 |
| RNAscope ® Probe - HPV93 | 421811 |
| RNAscope ® Probe - HPV96 | 421821 |
| RNAscope ® Probe - V-MusPV-L2 | 519371 |
| RNAscope ® Negative Control Probe - DapB | 310043 |
| RNAscope ® Positive Control Probe - Hs-PPIB | 313901 |

Next, we performed β-HPV RNAish on skin cancers from our immunosuppressed and immunocompetent patients (Table 3).

TABLE 3
Demographics of skin cancers used in β-HPV RNAish assays.
Tumors were stratified based on immune status of the
host then matched based on cancer type (BCC or SCC).

| | | |
|---|---|---|
| Immuno- | Immuno- | |
| suppressed* | competent | P |
| (n = 38) | (n = 32) | value** |

| | |
|---|---|
| Skin cancer, n | 0.6543 |

| | | |
|---|---|---|
| BCC | 2 | 3 |
| SCC*** | 36 | 29 |

| | | | | | |
|---|---|---|---|---|---|
| Age, mean (SD), y | 67 | (10) | 68 | (10) | 0.8911 |

| | | |
|---|---|---|
| (Range) | (41-87) | (49-91) |

| | | | | | |
|---|---|---|---|---|---|
| Gender, n (%) | | | | | 0.5885 |
| Male | 27 | (71) | 25 | (78) | |
| Female | 11 | (29) | 7 | (22) | |
| Anatomical sites of | | | | | 0.1829 |
| skin cancer, n (%) | | | | | |
| Head and neck | 13 | (34) | 19 | (59) | |
| Trunk | 8 | (21) | 5 | (16) | |
| Upper extremities | 10 | (26) | 5 | (16) | |
| Lower extremities | 7 | (19) | 3 | (9) | |

*The majority of immunosuppressed skin cancer samples were from organ transplant recipients.
**two-tailed fisher's exact test was used as the test of significance for skin cancer anatomical distribution outcomes. Pearson's χ² tests were used for other categorical variables.
***well-differentiated early invasive SCC and SCC in situ (SCCIS) samples are included in the study.

β-HPV RNA was detectable in the wart, hypertrophic actinic keratosis associated with wart and SCC from an immunosuppressed patient (FIG. 3a). β-HPV RNA expression was reduced in cancer cells compared to the adjacent normal skin cells of the immunosuppressed patient (FIG. 3a). β-HPV RNA expression was largely absent in the cancer cells of a SCC from an immunocompetent patient while a low level of β-HPV RNA expression was present in the adjacent normal skin (FIG. 3a). β-HPV RNAish signal counts across the skin cancers revealed a significant reduction in β-HPV RNA expression in cancer cells compared to adjacent normal skin keratinocytes among immunocompetent and immunosuppressed patients (p<0.001, FIG. 3b). Skin cancer cells in immunosuppressed patients had significantly more viral transcripts compared to skin cancer cells in immunocompetent patients (FIG. 15A). In addition, immunosuppressed patients' skin lesions had significantly higher β-HPV RNA expression across the tissue compared to the skin lesions and normal facial skin samples from immunocompetent patients (FIGS. 15B-C). β-HPV RNA expression was detectable in a significantly higher number of basal keratinocytes in the normal skin of the immunosuppressed compared to immunocompetent patients (FIGS. 16A-B). To examine β-HPV viral load at a subcellular level on the histological sections, we utilized a pool of μ-HPV DNA in situ hybridization (DNAish) probes that detect the sense strand of E6/7 genes from 25 β-HPV types. β-HPV DNAish revealed the subcellular localization of μ-HPV viral DNA in the human skin (FIG. 17). β-HPV DNAish showed high viral load in a wart, hypertrophic actinic keratosis associated with wart and SCC from an immunosuppressed patient (FIG. 18A). β-HPV viral load was reduced in the cancer cells compared to the adjacent normal skin of the immunosuppressed patient (p<0.05, FIG. 18B). The reduction in β-HPV viral load in cancer cells when compared to adjacent normal keratinocytes was more pronounced in the lesions of immunocompetent patients (p<0.01, FIG. 18C). The higher viral activity and load in the skin cancers of immunosuppressed patients correlated with significantly fewer tumor- and skin-infiltrating CD8+T and CD103+CD8+ TRM cells in their skin cancers compared with samples from immunocompetent patients (FIGS. 19A-C).

Finally, we examined whether the normal skin from a sun-damaged site contains T cells specific to β-HPV. T cells isolated from the normal facial skin of immunocompetent adults were exposed to peptides from E7 proteins of R genus types HPV5, 8, 9, 20 and 38 (Table 4). Skin-derived CD8⁺ cytotoxic T lymphocytes (CTLs) exposed to β-HPV peptides (test) or Phorbol myristate acetate and ionomycin (PMA/Ion; positive control) became activated as indicated by a significant increase in CD69⁺ and CD137⁺ CD69⁺ CTLs compared to the negative control (FIGS. 3C-D, 19D). Consistent with their activated state, we detected more CTL degranulation (CD107a⁺ CD8⁺ T cells) after exposure to β-HPV peptides compared to negative control (FIG. 19D). In contrast to β-TPV peptides, high-risk HIPV16 E7 peptides did not activate skin-derived CTLs (FIGS. 3C-D and FIG. 19D).

TABLE 4
Amino acid sequences of β-HPV and HPV16 E7
peptides used for in vitro T cell
stimulation assay. *HPV16 E7 peptides was used
as negative control, product code: PM-HPV16-E7.

| Strain | SEQ ID NO: |
|---|---|
| HPV5 E7 | |
| MIGKEVTVQDIILEL | 35. |
| EVTVQDIILELSEVQ | 36. |
| QDIILELSEVQPEVL | 37. |
| ELSEVQPEVLPVDLF | 38. |
| QPEVLPVDLFCEEEL | 39. |
| PVDLFCEEELPNEQE | 40. |
| FCEEELPNEQETEEE | 41. |
| LPNEQETEEEPDIER | 42. |
| QETEEEPDIERISYK | 43. |
| EEEPDIERISYKVIA | 44. |
| IERISYKVIAPCGCR | 45. |
| YKVIAPCGCRHCEVK | 46. |
| APCGCRHCEVKLRIF | 47. |
| RHCEVKLRIFVHATE | 48. |
| KLRIFVHATEFGIRA | 49. |
| VHATEFGIRAFQQLL | 50. |
| FGIRAFQQLLTGDLQ | 51. |
| FQQLLTGDLQLLCPD | 52. |
| LTGDLQLLCPDCRGN | 53. |
| QLLCPDCRGNCKHDG | 54. |
| LLCPDCRGNCKHDGS | 55. |
| HPV20 E7 | |
| MIGKEVTLQDIVLEL | 56. |
| EVTLQDIVLELNELQ | 57. |
| QDIVLELNELQPEVQ | 58. |
| ELNELQPEVQPVDLF | 59. |
| QPEVQPVDLFCEEEL | 60. |
| PVDLFCEEELPNEQQ | 61. |
| CEEELPNEQQEREEE | 62. |
| PNEQQEREEEPQIER | 63. |
| QEREEEPQIERASYK | 64. |
| EEEPQIERASYKVVA | 65. |
| PQIERASYKVVAPCG | 66. |
| ASYKVVAPCGCCKVK | 67. |
| VVAPCGCCKVKLRIF | 68. |
| GCCKVKLRIFISATE | 69. |
| KLRIFISATEFAIRS | 70. |
| ISATEFAIRSFQQLL | 71. |
| FAIRSFQQLLIDELQ | 72. |
| FQQLLIDELQLLCPD | 73. |
| LIDELQLLCPDCRGN | 74. |
| QLLCPDCRGNCKHGG | 75. |
| LLCPDCRGNCKHGGS | 76. |
| HPV8 E7 | |
| MIGKEVTVQDFVLKL | 77. |
| EVTVQDFVLKLSEIQ | 78. |
| QDFVLKLSEIQPEVL | 79. |
| KLSEIQPEVLPVDLL | 80. |
| QPEVLPVDLLCEEEL | 81. |
| PVDLLCEEELPNEQE | 82. |
| CEEELPNEQETEEEL | 83. |
| PNEQETEEELDIERT | 84. |
| QETEEELDIERTVFK | 85. |
| EEELDIERTVFKIVA | 86. |
| IERTVFKIVAPCGCS | 87. |
| FKIVAPCGCSCCQVK | 88. |
| APCGCSCCQVKLRLF | 89. |
| SCCQVKLRLFVNATD | 90. |
| KLRLFVNATDSGIRT | 91. |
| VNATDSGIRTFQELL | 92. |
| SGIRTFQELLFRDLQ | 93. |
| FQELLFRDLQLLCPE | 94. |
| LFRDLQLLCPECRGN | 95. |
| QLLCPECRGNCKHGG | 96. |
| HPV38 E7 | |
| MIGKQATLRDIVLEE | 97. |
| KQATLRDIVLEELVQ | 98. |
| RDIVLEELVQPIDLH | 99. |
| EELVQPIDLHCHEEL | 100. |
| PIDLHCHEELPDLPE | 101. |
| CHEELPDLPEDIEAS | 102. |
| PDLPEDIEASVVEEE | 103. |
| EDIEASVVEEEPAYT | 104. |
| EASVVEEEPAYTPYK | 105. |
| VEEEPAYTPYKIIVL | 106. |
| AYTPYKIIVLCGGCE | 107. |
| KIIVLCGGCEVRLKL | 108. |
| CGGCEVRLKLYVWAT | 109. |
| VRLKLYVWATDAGIR | 110. |
| LYVWATDAGIRNLQD | 111. |
| TDAGIRNLQDCLLGD | 112. |
| IRNLQDCLLGDVRLL | 113. |
| DCLLGDVRLLCPTCR | 114. |
| DVRLLCPTCREDIRN | 115. |
| LLCPTCREDIRNGGR | 116. |
| HPV9 E7 | |
| MIGKEATIPEVVLEL | 117. |
| ATIPEVVLELQELVQ | 118. |
| VVLELQELVQPTADL | 119. |
| QELVQPTADLHCYEE | 120. |
| QPTADLHCYEELTEE | 121. |
| LHCYEELTEEPAEEE | 122. |
| EELTEEPAEEEQCLT | 123. |
| TEEPAEEEQCLTPYK | 124. |
| AEEEQCLTPYKIVAG | 125. |
| CLTPYKIVAGCGCGA | 126. |
| KIVAGCGCGARLRLY | 127. |
| CGCGARLRLYVLATN | 128. |
| RLRLYVLATNLGIRA | 129. |
| VLATNLGIRAQQELL | 130. |
| LGIRAQQELLLGDIQ | 131. |
| QQELLLGDIQLVCPE | 132. |
| LGDIQLVCPECRGRL | 133. |
| IQLVCPECRGRLRHE | 134. |
| HPV16 E7* | |
| MHGDTPTLHEYMLDL | 135. |
| TPTLHEYMLDLQPET | 136. |
| HEYMLDLQPETTDLY | 137. |
| LDLQPETTDLYCYEQ | 138. |
| PETTDLYCYEQLNDS | 139. |
| DLYCYEQLNDSSEEE | 140. |
| YEQLNDSSEEEDEID | 141. |
| NDSSEEEDEIDGPAG | 142. |
| EEEDEIDGPAGQAEP | 143. |
| EIDGPAGQAEPDRAH | 144. |
| PAGQAEPDRAHYNIV | 145. |
| AEPDRAHYNIVTFCC | 146. |
| RAHYNIVTFCCKCDS | 147. |
| NIVTFCCKCDSTLRL | 148. |
| FCCKCDSTLRLCVQS | 149. |
| CDSTLRLCVQSTHVD | 150. |
| LRLCVQSTHVDIRTL | 151. |
| VQSTHVDIRTLEDLL | 152. |
| HVDIRTLEDLLMGTL | 153. |
| RTLEDLLMGTLGIVC | 154. |
| DLLMGTLGIVCPICS | 155. |
| MGTLGIVCPICSQKP | 156. |

To further develop the vaccine strategy, we use long overlapping peptides from MmuPV1, E1, E2, E6 and E7 proteins together with poly-ICLC adjuvant to vaccinate the Wt mice after colonization with MmuPV1. Next, we determine whether this vaccination reduces the skin cancer risk upon exposure to carcinogens. In addition, we study the efficacy of β-HPV multipeptide vaccine (peptides from Eli, L2, L4, L6 and L7 proteins of HPVs 5, 8, 9, 17, 20, 38, 50, 75, 80, and 151) in inducing immune response against skin cancer by injecting it subcutaneously at the site early squamous cell carcinomas in high-risk patients under an IRB-approved protocol. This study enables a trial to examine the vaccine application in high-risk patients for cancer prevention, for example the use of the vaccine in solid organ transplant recipients before they receive transplantation.

To identify the signals that lead to papillomavirus antigen presentation to T cells after abnormal proliferation of keratinocytes, we performed RNA sequencing (RNA-seq) on skin warts, MmuPV1-infected DMBA-UV-treated skin and tumors, and sham-infected DMBA-UV-treated skin and tumors of SKH-1 mice (FIGS. 20a-C). Among the 20 genes that were upregulated in both MmuPV1-induced warts and DMBA-UV-induced tumors (from both MmuPV1- and sham-infected groups) compared with skin (also from both groups), there were several immune-related genes including the damage-associated molecular pattern (DAMP) genes S100a8 and S100a9 (FIG. 20C). In human SCCs and warts, we confirmed the induction of S100 genes compared with normal skin and with seborrheic keratosis, a benign skin growth, in which S100A8 and S100A9 genes were downregulated compared with normal skin (FIGS. 20D-F).

### Example 2. Live HPV Vaccines and Live Attenuated HPV Vaccines

An in vitro culture system is used to expand cutaneotropic HPVs. Commensal HPVs are isolated, e.g., from warts of the adult immunosuppressed patients. Next, the purified virus is transferred to an organotypic raft culture model using Human Primary Keratinocytes (low passage rather than immortalized cell lines (Bienkowska-Haba et al., PLoS Pathog. 2018; 14(3):e1006846)). The difficulty in transfecting HPV genome into keratinocytes is resolved by using the extracellular matrix (ECM)-to-cell infection method as HPVs preferentially bind in vivo and in vitro to the basement membrane and the ECM secreted by keratinocytes (Richards et al., Viruses. 2014; 6(12):4856-79. Epub 2014/12/1). This involves the seeding of the cells on the surface of a collagen gel and later transferring this gel onto a stainless-steel grid with culture medium as to create an air-to-medium interface.

Mutation of the gene encoding for the E6 protein of commensal HPVs at its binding site to the LXXLL domain of MAML-1 allows for the development of a live attenuated virus that is safe for use in vaccine. The complete HPV genome is inserted into a bacterial artificial chromosome (BAC) for stable maintenance of the HPV genome within Escherichia coli and to introduce mutation into E6 at its binding site to MAML-1. The BAC sequence is flanked by loxP sites to allow for removal of the bacterial sequences from the viral genome by Cre recombination prior to transfer into human cells. Stepwise mutagenesis of the E6 proteins (as was done for MAML1 in Tan et al., Proceedings of the National Academy of Sciences of the United States of America. 2012; 109(23):E1473-80. Epub 2012/05/024). The recombinant viral genome is then introduced into Human Primary Keratinocytes. After transfection, the cells are differentiated and grown using the organotypic culture model, which supports the full HPV life cycle, and the effect of each mutation on binding of the E6 protein to MAML1 is determined.

### REFERENCES

1 Grulich, A. E., van Leeuwen, M. T., Falster, M. O. & Vajdic, C. M. Incidence of cancers in people with HIV/AIDS compared with immunosuppressed transplant recipients: a meta-analysis. Lancet 370, 59-67, doi:10.1016/S0140-6736(07)61050-2 (2007).
2 Wang, J., Aldabagh, B., Yu, J. & Arron, S. T. Role of human papillomavirus in cutaneous squamous cell carcinoma: a meta-analysis. Journal of the American Academy of Dermatology 70, 621-629, doi:10.1016/j.jaad.2014.01.857 (2014).
3 Forslund, O. et al. Cutaneous human papillomaviruses found in sun-exposed skin: Beta-papillomavirus species 2 predominates in squamous cell carcinoma. The Journal of infectious diseases 196, 876-883, doi:10.1086/521031 (2007).
4 Arron, S. T., Ruby, J. G., Dybbro, E., Ganem, D. & Derisi, J. L. Transcriptome sequencing demonstrates that human papillomavirus is not active in cutaneous squamous cell carcinoma. The Journal of investigative dermatology 131, 1745-1753, doi:10.1038/jid.2011.91 (2011).
5 Chockalingam, R., Downing, C. & Tyring, S. K. Cutaneous Squamous Cell Carcinomas in Organ Transplant Recipients. Journal of clinical medicine 4, 1229-1239, doi:10.3390/jcm4061229 (2015).
6 Ingle, A. et al. Novel laboratory mouse papillomavirus (MusPV) infection. Veterinary pathology 48, 500-505, doi:10.1177/0300985810377186 (2011).
7 Handisurya, A. et al. Strain-specific properties and T cells regulate the susceptibility to papilloma induction by Mus musculus papillomavirus 1. PLoS Pathog 10, e1004314, doi:10.1371/journal.ppat.1004314 (2014).
8 Cakir, B. O., Adamson, P. & Cingi, C. Epidemiology and economic burden of nonmelanoma skin cancer. Facial Plast Surg Clin North Am 20, 419-422, doi:10.1016/j.fsc.2012.07.004 (2012).
9 Rogers, H. W., Weinstock, M. A., Feldman, S. R. & Coldiron, B. M. Incidence Estimate of Nonmelanoma Skin Cancer (Keratinocyte Carcinomas) in the U.S. Population, 2012. JAMA dermatology 151, 1081-1086, doi:10.1001/jamadermatol.2015.1187 (2015).
10 Eigentler, T. K. et al. Survival of Patients with Cutaneous Squamous Cell Carcinoma: Results of a Prospective Cohort Study. The Journal of investigative dermatology 137, 2309-2315, doi:10.1016/j.jid.2017.06.025 (2017).
11 Clayman, G. L. et al. Mortality risk from squamous cell skin cancer. J Clin Oncol 23, 759-765, doi:10.1200/JCO.2005.02.155 (2005).
12 Nehal, K. S. & Bichakjian, C. K. Update on Keratinocyte Carcinomas. The New England journal of medicine 379, 363-374, doi:10.1056/NEJMra1708701 (2018).
13 Guy, G. P., Jr., Machlin, S. R., Ekwueme, D. U. & Yabroff, K. R. Prevalence and costs of skin cancer treatment in the U.S., 2002-2006 and 2007-2011. Am J Prev Med 48, 183-187, doi:10.1016/j.amepre.2014.08.036 (2015).
14 Howley, P. M. & Pfister, H. J. Beta genus papillomaviruses and skin cancer. Virology 479-480, 290-296, doi:10.1016/j.virol.2015.02.004 (2015).
15 de Koning, M. N. et al. Betapapillomaviruses frequently persist in the skin of healthy individuals. J Gen Virol 88, 1489-1495, doi:10.1099/vir.0.82732-0 (2007).
16 Asgari, M. M. et al. Detection of human papillomavirus DNA in cutaneous squamous cell carcinoma among immunocompetent individuals. The Journal of investigative dermatology 128, 1409-1417, doi:10.1038/sj.jid.5701227 (2008).
17 Weissenborn, S. J. et al. Human papillomavirus-DNA loads in actinic keratoses exceed those in non-melanoma skin cancers. The Journal of investigative dermatology 125, 93-97, doi:10.1111/j.0022-202X.2005.23733.x (2005).
18 Forslund, O. et al. High prevalence of cutaneous human papillomavirus DNA on the top of skin tumors but not in “Stripped” biopsies from the same tumors. The Journal of investigative dermatology 123, 388-394, doi:10.1111/j.0022-202X.2004.23205.x (2004).
19 Hufbauer, M. & Akgul, B. Molecular Mechanisms of Human Papillomavirus Induced Skin Carcinogenesis. Viruses 9, doi:10.3390/v9070187 (2017).
20 Abel, E. L., Angel, J. M., Kiguchi, K. & DiGiovanni, J. Multi-stage chemical carcinogenesis in mouse skin: fundamentals and applications. Nature protocols 4, 1350-1362 (2009).
21 Brouxhon, S. et al. Deletion of prostaglandin E2 EP2 receptor protects against ultraviolet-induced carcinogenesis, but increases tumor aggressiveness. The Journal of investigative dermatology 127, 439-446, doi:10.1038/sj.jid.5700547 (2007).
22 Wang, J. W. et al. Immunologic Control of Mus musculus Papillomavirus Type 1. PLoS Pathog 11, e1005243, doi:10.1371/journal.ppat.1005243 (2015).
23 Uberoi, A., Yoshida, S., Frazer, I. H., Pitot, H. C. & Lambert, P. F. Role of Ultraviolet Radiation in Papillomavirus-Induced Disease. PLoS Pathog 12, e1005664, doi:10.1371/journal.ppat.1005664 (2016).
24 Johnson, M. T. & Roberts, J. Skin conditions and related need for medical care among persons 1-74 years. United States, 1971-1974. Vital Health Stat 11, i-v, 1-72 (1978).
25 Inman, G. J. et al. The genomic landscape of cutaneous SCC reveals drivers and a novel azathioprine associated mutational signature. Nature communications 9, 3667, doi:10.1038/s41467-018-06027-1 (2018).
26 Goh, G. et al. Mutational landscape of MCPyV-positive and MCPyV-negative Merkel cell carcinomas with implications for immunotherapy. Oncotarget 7, 3403-3415, doi:10.18632/oncotarget.6494 (2016).
27 Nghiem, P. T. et al. PD-1 Blockade with Pembrolizumab in Advanced Merkel-Cell Carcinoma. The New England journal of medicine 374, 2542-2552, doi:10.1056/NEJMoa1603702 (2016).
28 Joh, J. et al. T cell-mediated antitumor immune response eliminates skin tumors induced by mouse papillomavirus, MmuPV1. Exp Mol Pathol 103, 181-190, doi:10.1016/j.yexmp.2017.09.003 (2017).
29 Martincorena, I. et al. Tumor evolution. High burden and pervasive positive selection of somatic mutations in normal human skin. Science (New York, N.Y 348, 880-886, doi:10.1126/science.aaa6806 (2015).
30 Meyers, J. M., Uberoi, A., Grace, M., Lambert, P. F. & Munger, K. Cutaneous HPV8 and MmuPV1 E6 Proteins Target the NOTCH and TGF-beta Tumor Suppressors to Inhibit Differentiation and Sustain Keratinocyte Proliferation. PLoS Pathog 13, e1006171, doi:10.1371/journal.ppat.1006171 (2017).
31 Viarisio, D. et al. Beta HPV38 oncoproteins act with a hit-and-run mechanism in ultraviolet radiation-induced skin carcinogenesis in mice. PLoS Pathog 14, e1006783, doi:10.1371/journal.ppat.1006783 (2018).
32 Hasche, D. et al. The interplay of UV and cutaneous papillomavirus infection in skin cancer development. PLoS Pathog 13, e1006723, doi:10.1371/journal.ppat.1006723 (2017).
33 Viarisio, D. et al. E6 and E7 from beta HPV38 cooperate with ultraviolet light in the development of actinic keratosis-like lesions and squamous cell carcinoma in mice. PLoS Pathog 7, e1002125, doi:10.1371/journal.ppat.1002125 (2011).
34 Herrero, R. Human papillomavirus (HPV) vaccines: limited cross-protection against additional HPV types. The Journal of infectious diseases 199, 919-922, doi:10.1086/597308 (2009).
35 Tirosh, O. et al. Expanded skin virome in DOCK8-deficient patients. Nature medicine, doi:10.1038/s41591-018-0211-7 (2018).
36 Joh, J. et al. Molecular diagnosis of a laboratory mouse papillomavirus (MusPV). Exp Mol Pathol 93, 416-421, doi:10.1016/j.yexmp.2012.07.001 (2012).
37 Jiang, R. T. et al. Spontaneous and Vaccine-Induced Clearance of Mus Musculus Papillomavirus 1 Infection. Journal of virology 91, doi:10.1128/JVI.00699-17 (2017).
38 Chen, B. J., Cui, X., Sempowski, G. D., Liu, C. & Chao, N. J. Transfer of allogeneic CD62L-memory T cells without graft-versus-host disease. Blood 103, 1534-1541, doi: 10.1182/blood-2003-08-2987 (2004).
39 D'Orazio, J. A. et al. Topical drug rescue strategy and skin protection based on the role of Mclr in UV-induced tanning. Nature 443, 340-344, doi:10.1038/nature05098 (2006).
40 Technology Planning and Management Corporation. Report on Carcinogens Background Document for Broad-Spectrum Ultraviolet (UV) Radiation and UVA, and UVB, and UVC. (Technology Planning and Management Corporation 2000).
41 Joh, J., Jenson, A. B., Ingle, A., Sundberg, J. P. & Ghim, S. J. Searching for the initiating site of the major capsid protein to generate virus-like particles for a novel laboratory mouse papillomavirus. Exp Mol Pathol 96, 155-161, doi:10.1016/j.yexmp.2013.12.009 (2014).
42 Wang, F. et al. RNAscope: a novel in situ RNA analysis platform for formalin-fixed, paraffin-embedded tissues. The Journal of molecular diagnostics: JMD 14, 22-29, doi:10.1016/j.jmoldx.2011.08.002 (2012).
43 Dang, C. et al. E6/E7 expression of human papillomavirus types in cutaneous squamous cell dysplasia and carcinoma in immunosuppressed organ transplant recipients. The British journal of dermatology 155, 129-136, doi:10.1111/j.1365-2133.2006.07378.x (2006).
44 Watanabe, R. et al. Human skin is protected by four functionally and phenotypically discrete populations of resident and recirculating memory T cells. Science translational medicine 7, 279ra239, doi:10.1126/scitranslmed.3010302 (2015).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
a plurality of (i) antigenic peptides, each comprising a sequence of 9-30 amino acids derived from proteins from commensal human papilloma viruses, or (ii) live or live-attenuated commensal human papilloma viruses; and
a T cell adjuvant that increases T cell response to the antigenic peptides.

2. The composition of claim 1, wherein the commensal human papilloma viruses are low risk α-HPV, β-HPV, γ-HPV, and/or μ-HPV strains.

3. The composition of claim 1, wherein the commensal human papilloma viruses are low risk α-HPV, β-HPV, γ-HPV, and/or μ-HPV strains listed in Table A.

4. The composition of claim 1, wherein the plurality of antigenic peptides comprises peptides derived from one or more E1, E2, E4, E5, E6 or E7 proteins.

5. The composition of claim 1, wherein the plurality of antigenic peptides comprises peptides derived from proteins from a plurality of commensal human papilloma viruses.

6. The composition of claim 5, comprising at least 200 peptides each having a unique sequences.

7. The composition of claim 6, comprising a plurality of peptides for each unique sequence.

8. A composition comprising:
a plurality of antigenic proteins from commensal human papilloma viruses, preferably in virus-like particles; and
a T cell adjuvant that increases T cell response to the antigenic proteins.

9. The composition of claim 8, wherein the plurality of antigenic proteins comprise one or more E1, E2, E4, E5, E6 or E7 proteins.

10. The composition of claim 8, wherein the commensal human papilloma viruses are low risk α-HPV, β-HPV, γ-HPV, and/or μ-HPV strains.

11. The composition of claim 8, wherein the commensal human papilloma viruses are low risk α-HPV, β-HPV, γ-HPV, and/or μ-HPV strains listed in Table A.

12. A composition comprising a plurality of nucleic acids encoding (i) a plurality of antigenic peptides, each comprising a sequence of 9-30 amino acids derived from proteins from commensal human papilloma viruses; or (ii) a plurality of antigenic proteins from commensal human papilloma viruses; and
a T cell adjuvant that increases T cell response to the antigenic peptides.

13. The composition of claim 12, wherein the plurality of antigenic proteins comprise one or more E1, E2, E4, E5, E6 or E7 proteins.

14. The composition of claim 12, wherein the commensal human papilloma viruses are low risk α-HPV, β-HPV, γ-HPV, and/or μ-HPV strains.

15. The composition of claim 12, wherein the commensal human papilloma viruses are low risk α-HPV, β-HPV, γ-HPV, and/or μ-HPV strains listed in Table A.

16. The composition of claim 12, comprising one or more viral vectors engineered to express the plurality of proteins or antigenic peptides.

17. The composition of claim 12, wherein the viral vectors are selected from the group consisting of recombinant retroviruses, adenovirus, adeno-associated virus, alphavirus, and lentivirus.

18. The composition of claim 1, wherein the T cell adjuvant comprises one or more of nanoparticles that enhance T cell response; poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminium salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant).

19. The composition of claim 1, wherein the T cell adjuvant comprises topical resiquimod or topical imiquimod or topical 5-fluorouracil or topical calcipotriene (calcipotriol) or their combination, optionally calcipotriene in combination with 5-fluorouracil.

20. A method of treating, or reducing the risk of developing, skin cancer in a subject, the method comprising administering to the subject an effective amount of the composition of claim 1.

21. The method of claim 20, wherein the subject has an increased risk of developing skin cancer or is immunocompromised.

22. The method of claim 21, wherein the subject is immunocompromised as a result of aging or an acquired immunodeficiency or an organ transplant.

23.-25. (canceled)


SEQUENCE LISTING (156 sequences)

SEQ ID NO: 1 - DNA, 21 bp, Artificial Sequence
  Description of Artificial Sequence Synthetic primer
gagctctttgttactgttgtc

SEQ ID NO: 2 - DNA, 19 bp, Artificial Sequence
  Description of Artificial Sequence Synthetic primer
atcctctctttccttgggc

SEQ ID NO: 3 - DNA, 20 bp, Artificial Sequence
  Description of Artificial Sequence Synthetic primer
gccgaacaccaacagaaact

SEQ ID NO: 4 - DNA, 21 bp, Artificial Sequence
  Description of Artificial Sequence Synthetic primer
aacaatcaatcacagggatgc

SEQ ID NO: 5 - DNA, 22 bp, Artificial Sequence
  Description of Artificial Sequence Synthetic primer
agcttatttggacagaggagga

SEQ ID NO: 6 - DNA, 20 bp, Artificial Sequence
  Description of Artificial Sequence Synthetic primer
gtgcagacgcataagcacag

SEQ ID NO: 7 - DNA, 22 bp, Artificial Sequence
  Description of Artificial Sequence Synthetic primer
tgcagttaatttggactgagga

SEQ ID NO: 8 - DNA, 20 bp, Artificial Sequence
  Description of Artificial Sequence Synthetic primer
attcaaactgcgctgtagca

SEQ ID NO: 9 - DNA, 23 bp, Artificial Sequence
  Description of Artificial Sequence Synthetic primer
tttggcggctggaggaggtcaca

SEQ ID NO: 10 - DNA, 21 bp, Artificial Sequence
  Description of Artificial Sequence Synthetic primer
atcgccacctaccgccgcctg

SEQ ID NO: 11 - DNA, 13 bp, Artificial Sequence
  Description of Artificial Sequence Synthetic probe
accaggtcttctg

SEQ ID NO: 12 - DNA, 20 bp, Artificial Sequence
  Description of Artificial Sequence Synthetic primer
gcatgtctcttgtcagctgt

SEQ ID NO: 13 - DNA, 22 bp, Artificial Sequence
  Description of Artificial Sequence Synthetic primer
cgtcgatgatagagttcaaggc

SEQ ID NO: 14 - DNA, 16 bp, Artificial Sequence
  Description of Artificial Sequence Synthetic probe
aattccccctggttca

SEQ ID NO: 15 - DNA, 22 bp, Artificial Sequence
  Description of Artificial Sequence Synthetic primer
caacaccttccaccaatactct

SEQ ID NO: 16 - DNA, 23 bp, Artificial Sequence
  Description of Artificial Sequence Synthetic primer
cctccatgatgtgttctatgacc

SEQ ID NO: 17 - DNA, 16 bp, Artificial Sequence
  Description of Artificial Sequence Synthetic probe
gtcctttggtgagagt

SEQ ID NO: 18 - DNA, 20 bp, Artificial Sequence
  Description of Artificial Sequence Synthetic primer
acagatcatgagccatcagc

SEQ ID NO: 19 - DNA, 21 bp, Artificial Sequence
  Description of Artificial Sequence Synthetic primer
actgaattcctgagcatcctc

SEQ ID NO: 20 - DNA, 15 bp, Artificial Sequence
  Description of Artificial Sequence Synthetic probe
aggcagtcattgtcc

SEQ ID NO: 21 - DNA, 18 bp, Artificial Sequence
  Description of Artificial Sequence Synthetic primer
ctggagaggaggcagact

SEQ ID NO: 22 - DNA, 21 bp, Artificial Sequence
  Description of Artificial Sequence Synthetic primer
cttgttcttgaccaggctgta

SEQ ID NO: 23 - DNA, 18 bp, Artificial Sequence
  Description of Artificial Sequence Synthetic probe
caaccctaaacatccaaa

SEQ ID NO: 24 - DNA, 20 bp, Artificial Sequence
  Description of Artificial Sequence Synthetic primer
agccactttatgcttcctga

SEQ ID NO: 25 - DNA, 21 bp, Artificial Sequence
  Description of Artificial Sequence Synthetic primer
tggcatcaagggataaggaag

SEQ ID NO: 26 - DNA, 16 bp, Artificial Sequence
  Description of Artificial Sequence Synthetic probe
gtccctctggtcctgt

SEQ ID NO: 27 - DNA, 21 bp, Artificial Sequence
  Description of Artificial Sequence Synthetic primer
cctctgattggtgaatggtga

SEQ ID NO: 28 - DNA, 21 bp, Artificial Sequence
  Description of Artificial Sequence Synthetic primer
ggtgttaaattggcgaaagca

SEQ ID NO: 29 - DNA, 15 bp, Artificial Sequence
  Description of Artificial Sequence Synthetic probe
tgagctcgccagtga

SEQ ID NO: 30 - DNA, 22 bp, Artificial Sequence
  Description of Artificial Sequence Synthetic primer
cagccaatcttcattgctcaag

SEQ ID NO: 31 - DNA, 21 bp, Artificial Sequence
  Description of Artificial Sequence Synthetic primer
gaacaagtcatcctcattgcc

SEQ ID NO: 32 - DNA, 15 bp, Artificial Sequence
  Description of Artificial Sequence Synthetic probe
gctatagagcgtgcc

SEQ ID NO: 33 - DNA, 19 bp, Artificial Sequence
  Description of Artificial Sequence Synthetic primer
gcactaccactgctactgt

SEQ ID NO: 34 - DNA, 23 bp, Artificial Sequence
  Description of Artificial Sequence Synthetic primer
agttcagcaattacagcatcaac

SEQ ID NO: 35 - protein, 15 aa, Human papillomavirus
MIGKEVTVQDIILEL

SEQ ID NO: 36 - protein, 15 aa, Human papillomavirus
EVTVQDIILELSEVQ

SEQ ID NO: 37 - protein, 15 aa, Human papillomavirus
QDIILELSEVQPEVL

SEQ ID NO: 38 - protein, 15 aa, Human papillomavirus
ELSEVQPEVLPVDLF

SEQ ID NO: 39 - protein, 15 aa, Human papillomavirus
QPEVLPVDLFCEEEL

SEQ ID NO: 40 - protein, 15 aa, Human papillomavirus
PVDLFCEEELPNEQE

SEQ ID NO: 41 - protein, 15 aa, Human papillomavirus
FCEEELPNEQETEEE

SEQ ID NO: 42 - protein, 15 aa, Human papillomavirus
LPNEQETEEEPDIER

SEQ ID NO: 43 - protein, 15 aa, Human papillomavirus
QETEEEPDIERISYK

SEQ ID NO: 44 - protein, 15 aa, Human papillomavirus
EEEPDIERISYKVIA

SEQ ID NO: 45 - protein, 15 aa, Human papillomavirus
IERISYKVIAPCGCR

SEQ ID NO: 46 - protein, 15 aa, Human papillomavirus
YKVIAPCGCRHCEVK

SEQ ID NO: 47 - protein, 15 aa, Human papillomavirus
APCGCRHCEVKLRIF

SEQ ID NO: 48 - protein, 15 aa, Human papillomavirus
RHCEVKLRIFVHATE

SEQ ID NO: 49 - protein, 15 aa, Human papillomavirus
KLRIFVHATEFGIRA

SEQ ID NO: 50 - protein, 15 aa, Human papillomavirus
VHATEFGIRAFQQLL

SEQ ID NO: 51 - protein, 15 aa, Human papillomavirus
FGIRAFQQLLTGDLQ

SEQ ID NO: 52 - protein, 15 aa, Human papillomavirus
FQQLLTGDLQLLCPD

SEQ ID NO: 53 - protein, 15 aa, Human papillomavirus
LTGDLQLLCPDCRGN

SEQ ID NO: 54 - protein, 15 aa, Human papillomavirus
QLLCPDCRGNCKHDG

SEQ ID NO: 55 - protein, 15 aa, Human papillomavirus
LLCPDCRGNCKHDGS

SEQ ID NO: 56 - protein, 15 aa, Human papillomavirus
MIGKEVTLQDIVLEL

SEQ ID NO: 57 - protein, 15 aa, Human papillomavirus
EVTLQDIVLELNELQ

SEQ ID NO: 58 - protein, 15 aa, Human papillomavirus
QDIVLELNELQPEVQ

SEQ ID NO: 59 - protein, 15 aa, Human papillomavirus
ELNELQPEVQPVDLF

SEQ ID NO: 60 - protein, 15 aa, Human papillomavirus
QPEVQPVDLFCEEEL

SEQ ID NO: 61 - protein, 15 aa, Human papillomavirus
PVDLFCEEELPNEQQ

SEQ ID NO: 62 - protein, 15 aa, Human papillomavirus
CEEELPNEQQEREEE

SEQ ID NO: 63 - protein, 15 aa, Human papillomavirus
PNEQQEREEEPQIER

SEQ ID NO: 64 - protein, 15 aa, Human papillomavirus
QEREEEPQIERASYK

SEQ ID NO: 65 - protein, 15 aa, Human papillomavirus
EEEPQIERASYKVVA

SEQ ID NO: 66 - protein, 15 aa, Human papillomavirus
PQIERASYKVVAPCG

SEQ ID NO: 67 - protein, 15 aa, Human papillomavirus
ASYKVVAPCGCCKVK

SEQ ID NO: 68 - protein, 15 aa, Human papillomavirus
VVAPCGCCKVKLRIF

SEQ ID NO: 69 - protein, 15 aa, Human papillomavirus
GCCKVKLRIFISATE

SEQ ID NO: 70 - protein, 15 aa, Human papillomavirus
KLRIFISATEFAIRS

SEQ ID NO: 71 - protein, 15 aa, Human papillomavirus
ISATEFAIRSFQQLL

SEQ ID NO: 72 - protein, 15 aa, Human papillomavirus
FAIRSFQQLLIDELQ

SEQ ID NO: 73 - protein, 15 aa, Human papillomavirus
FQQLLIDELQLLCPD

SEQ ID NO: 74 - protein, 15 aa, Human papillomavirus
LIDELQLLCPDCRGN

SEQ ID NO: 75 - protein, 15 aa, Human papillomavirus
QLLCPDCRGNCKHGG

SEQ ID NO: 76 - protein, 15 aa, Human papillomavirus
LLCPDCRGNCKHGGS

SEQ ID NO: 77 - protein, 15 aa, Human papillomavirus
MIGKEVTVQDFVLKL

SEQ ID NO: 78 - protein, 15 aa, Human papillomavirus
EVTVQDFVLKLSEIQ

SEQ ID NO: 79 - protein, 15 aa, Human papillomavirus
QDFVLKLSEIQPEVL

SEQ ID NO: 80 - protein, 15 aa, Human papillomavirus
KLSEIQPEVLPVDLL

SEQ ID NO: 81 - protein, 15 aa, Human papillomavirus
QPEVLPVDLLCEEEL

SEQ ID NO: 82 - protein, 15 aa, Human papillomavirus
PVDLLCEEELPNEQE

SEQ ID NO: 83 - protein, 15 aa, Human papillomavirus
CEEELPNEQETEEEL

SEQ ID NO: 84 - protein, 15 aa, Human papillomavirus
PNEQETEEELDIERT

SEQ ID NO: 85 - protein, 15 aa, Human papillomavirus
QETEEELDIERTVFK

SEQ ID NO: 86 - protein, 15 aa, Human papillomavirus
EEELDIERTVFKIVA

SEQ ID NO: 87 - protein, 15 aa, Human papillomavirus
IERTVFKIVAPCGCS

SEQ ID NO: 88 - protein, 15 aa, Human papillomavirus
FKIVAPCGCSCCQVK

SEQ ID NO: 89 - protein, 15 aa, Human papillomavirus
APCGCSCCQVKLRLF

SEQ ID NO: 90 - protein, 15 aa, Human papillomavirus
SCCQVKLRLFVNATD

SEQ ID NO: 91 - protein, 15 aa, Human papillomavirus
KLRLFVNATDSGIRT

SEQ ID NO: 92 - protein, 15 aa, Human papillomavirus
VNATDSGIRTFQELL

SEQ ID NO: 93 - protein, 15 aa, Human papillomavirus
SGIRTFQELLFRDLQ

SEQ ID NO: 94 - protein, 15 aa, Human papillomavirus
FQELLFRDLQLLCPE

SEQ ID NO: 95 - protein, 15 aa, Human papillomavirus
LFRDLQLLCPECRGN

SEQ ID NO: 96 - protein, 15 aa, Human papillomavirus
QLLCPECRGNCKHGG

SEQ ID NO: 97 - protein, 15 aa, Human papillomavirus
MIGKQATLRDIVLEE

SEQ ID NO: 98 - protein, 15 aa, Human papillomavirus
KQATLRDIVLEELVQ

SEQ ID NO: 99 - protein, 15 aa, Human papillomavirus
RDIVLEELVQPIDLH

SEQ ID NO: 100 - protein, 15 aa, Human papillomavirus
EELVQPIDLHCHEEL

SEQ ID NO: 101 - protein, 15 aa, Human papillomavirus
PIDLHCHEELPDLPE

SEQ ID NO: 102 - protein, 15 aa, Human papillomavirus
CHEELPDLPEDIEAS

SEQ ID NO: 103 - protein, 15 aa, Human papillomavirus
PDLPEDIEASVVEEE

SEQ ID NO: 104 - protein, 15 aa, Human papillomavirus
EDIEASVVEEEPAYT

SEQ ID NO: 105 - protein, 15 aa, Human papillomavirus
EASVVEEEPAYTPYK

SEQ ID NO: 106 - protein, 15 aa, Human papillomavirus
VEEEPAYTPYKIIVL

SEQ ID NO: 107 - protein, 15 aa, Human papillomavirus
AYTPYKIIVLCGGCE

SEQ ID NO: 108 - protein, 15 aa, Human papillomavirus
KIIVLCGGCEVRLKL

SEQ ID NO: 109 - protein, 15 aa, Human papillomavirus
CGGCEVRLKLYVWAT

SEQ ID NO: 110 - protein, 15 aa, Human papillomavirus
VRLKLYVWATDAGIR

SEQ ID NO: 111 - protein, 15 aa, Human papillomavirus
LYVWATDAGIRNLQD

SEQ ID NO: 112 - protein, 15 aa, Human papillomavirus
TDAGIRNLQDCLLGD

SEQ ID NO: 113 - protein, 15 aa, Human papillomavirus
IRNLQDCLLGDVRLL

SEQ ID NO: 114 - protein, 15 aa, Human papillomavirus
DCLLGDVRLLCPTCR

SEQ ID NO: 115 - protein, 15 aa, Human papillomavirus
DVRLLCPTCREDIRN

SEQ ID NO: 116 - protein, 15 aa, Human papillomavirus
LLCPTCREDIRNGGR

SEQ ID NO: 117 - protein, 15 aa, Human papillomavirus
MIGKEATIPEVVLEL

SEQ ID NO: 118 - protein, 15 aa, Human papillomavirus
ATIPEVVLELQELVQ

SEQ ID NO: 119 - protein, 15 aa, Human papillomavirus
VVLELQELVQPTADL

SEQ ID NO: 120 - protein, 15 aa, Human papillomavirus
QELVQPTADLHCYEE

SEQ ID NO: 121 - protein, 15 aa, Human papillomavirus
QPTADLHCYEELTEE

SEQ ID NO: 122 - protein, 15 aa, Human papillomavirus
LHCYEELTEEPAEEE

SEQ ID NO: 123 - protein, 15 aa, Human papillomavirus
EELTEEPAEEEQCLT

SEQ ID NO: 124 - protein, 15 aa, Human papillomavirus
TEEPAEEEQCLTPYK

SEQ ID NO: 125 - protein, 15 aa, Human papillomavirus
AEEEQCLTPYKIVAG

SEQ ID NO: 126 - protein, 15 aa, Human papillomavirus
CLTPYKIVAGCGCGA

SEQ ID NO: 127 - protein, 15 aa, Human papillomavirus
KIVAGCGCGARLRLY

SEQ ID NO: 128 - protein, 15 aa, Human papillomavirus
CGCGARLRLYVLATN

SEQ ID NO: 129 - protein, 15 aa, Human papillomavirus
RLRLYVLATNLGIRA

SEQ ID NO: 130 - protein, 15 aa, Human papillomavirus
VLATNLGIRAQQELL

SEQ ID NO: 131 - protein, 15 aa, Human papillomavirus
LGIRAQQELLLGDIQ

SEQ ID NO: 132 - protein, 15 aa, Human papillomavirus
QQELLLGDIQLVCPE

SEQ ID NO: 133 - protein, 15 aa, Human papillomavirus
LGDIQLVCPECRGRL

SEQ ID NO: 134 - protein, 15 aa, Human papillomavirus
IQLVCPECRGRLRHE

SEQ ID NO: 135 - protein, 15 aa, Human papillomavirus
MHGDTPTLHEYMLDL

SEQ ID NO: 136 - protein, 15 aa, Human papillomavirus
TPTLHEYMLDLQPET

SEQ ID NO: 137 - protein, 15 aa, Human papillomavirus
HEYMLDLQPETTDLY

SEQ ID NO: 138 - protein, 15 aa, Human papillomavirus
LDLQPETTDLYCYEQ

SEQ ID NO: 139 - protein, 15 aa, Human papillomavirus
PETTDLYCYEQLNDS

SEQ ID NO: 140 - protein, 15 aa, Human papillomavirus
DLYCYEQLNDSSEEE

SEQ ID NO: 141 - protein, 15 aa, Human papillomavirus
YEQLNDSSEEEDEID

SEQ ID NO: 142 - protein, 15 aa, Human papillomavirus
NDSSEEEDEIDGPAG

SEQ ID NO: 143 - protein, 15 aa, Human papillomavirus
EEEDEIDGPAGQAEP

SEQ ID NO: 144 - protein, 15 aa, Human papillomavirus
EIDGPAGQAEPDRAH

SEQ ID NO: 145 - protein, 15 aa, Human papillomavirus
PAGQAEPDRAHYNIV

SEQ ID NO: 146 - protein, 15 aa, Human papillomavirus
AEPDRAHYNIVTFCC

SEQ ID NO: 147 - protein, 15 aa, Human papillomavirus
RAHYNIVTFCCKCDS

SEQ ID NO: 148 - protein, 15 aa, Human papillomavirus
NIVTFCCKCDSTLRL

SEQ ID NO: 149 - protein, 15 aa, Human papillomavirus
FCCKCDSTLRLCVQS

SEQ ID NO: 150 - protein, 15 aa, Human papillomavirus
CDSTLRLCVQSTHVD

SEQ ID NO: 151 - protein, 15 aa, Human papillomavirus
LRLCVQSTHVDIRTL

SEQ ID NO: 152 - protein, 15 aa, Human papillomavirus
VQSTHVDIRTLEDLL

SEQ ID NO: 153 - protein, 15 aa, Human papillomavirus
HVDIRTLEDLLMGTL

SEQ ID NO: 154 - protein, 15 aa, Human papillomavirus
RTLEDLLMGTLGIVC

SEQ ID NO: 155 - protein, 15 aa, Human papillomavirus
DLLMGTLGIVCPICS

SEQ ID NO: 156 - protein, 15 aa, Human papillomavirus
MGTLGIVCPICSQKP